(19) United States
(12) Patent Application Publication
(10) Pub. No.: US 2010/0198308 A1
(43) Pub. Date: Aug. 5, 2010

(54) CLOSED-LOOP NEUROSTIMULATION TO TREAT PULMONARY EDEMA

(75) Inventors: Xiaohong Zhou, Woodbury, MN (US); William T. Donofrio, Andover, MN (US); Paul G. Krause, Shoreview, MN (US); John E. Burnes, Coon Rapids, MN (US)

Correspondence Address: Medtronic, Inc., 710 Medtronic Parkway, Minneapolis, MN, 55432, US

(73) Assignee: Medtronic, Inc., Minneapolis, MN (US)

(21) Appl. No.: 12/696,914

(22) Filed: Jan. 29, 2010

Related U.S. Application Data
(60) Provisional application No. 61/148,550, filed on Jan. 30, 2009.

(51) Int. Cl. A61N 1/36

(52) U.S. Cl. 607/62

(57) ABSTRACT

Neurostimulation to mitigate lung wetness is delivered to a patient based on a sensed parameter indicative of lung wetness. The neurostimulation is configured to at least one of increase parasympathetic activity or decrease sympathetic activity within the patient. In some examples, a patient response to the neurostimulation therapy may be detected to modify the neurostimulation therapy. The patient response may include, for example, changes in the contractility of a heart of the patient, changes in the heart rate, heart rate variability or blood pressure of the patient, changes in a bladder size of the patient, changes in bladder functional activity of the patient, changes in urine flow, changes in lung function, changes in lung composition, or changes in the nerve activity of the patient.

This application claims the benefit of U.S. Provisional Application No. 61/148,550, entitled, “CLOSED-LOOP NEUROSTIMULATION TO TREAT PULMONARY EDEMA,” and filed on Jan. 30, 2009, the entire content of which is incorporated herein by reference.

### TECHNICAL FIELD

The disclosure relates to medical devices and, more particularly, medical devices that deliver electrical stimulation.

### BACKGROUND

A wide variety of implantable medical devices that deliver a therapy or monitor a physiologic condition of a patient have been clinically implanted or proposed for clinical implantation in patients. Some implantable medical devices may employ one or more elongated electrical leads and/or sensors. Such implantable medical devices may deliver therapy or monitor the heart, muscle, nerve, brain, stomach or other organs. In some cases, implantable medical devices deliver electrical stimulation therapy and/or monitor physiological signals via one or more electrodes or sensor elements, which may be included as part of one or more elongated implantable medical leads. Implantable medical leads may be configured to allow electrodes or sensors to be positioned at desired locations for delivery of stimulation or sensing electrical depolarizations. For example, electrodes or sensors may be located at a distal portion of the lead. A proximal portion of the lead may be coupled to an implantable medical device housing, which may contain electronic circuitry such as stimulation generation and/or sensing circuitry.

For example, implantable cardiac devices, such as cardiac pacemakers or implantable cardioverter defibrillators, provide therapeutic stimulation to the heart by delivering electrical therapy signals such as pulses or shocks for pacing, cardioversion or defibrillation pulses via electrodes of one or more implantable leads. In some cases, an implantable cardiac device may sense intrinsic depolarizations of the heart, and control the delivery of therapeutic stimulation to the heart based on the sensing. When an abnormal rhythm of the heart is detected, such as bradycardia, tachycardia or fibrillation, an appropriate electrical therapy (e.g., in the form of pulses) may be delivered to restore the normal rhythm. For example, in some cases, an implantable medical device may deliver pacing, cardioversion or defibrillation signals to the heart of the patient upon detecting ventricular tachycardia, and deliver cardioversion or defibrillation therapy to a patient's heart upon detecting ventricular fibrillation. Some proposed medical device systems include a neurostimulator in addition to the implantable cardiac device.

### SUMMARY

In general, the disclosure is directed to delivering neurostimulation to a patient based on a sensed parameter indicative of lung wetness, which may be an indicator of heart failure. In some patients, such as patients with heart failure, mitigating lung wetness may improve cardiac function. Therefore, delivering neurostimulation that is configured to mitigate lung wetness may complement cardiac rhythm therapy, e.g., pacing, cardioversion, and/or defibrillation therapy. Likewise, improving cardiac function (e.g., increasing cardiac output) or kidney function (e.g., increasing fluid excretion) may help mitigate lung wetness and the need for neurostimulation. As described herein, neurostimulation may be configured to mitigate lung wetness by at least one of increasing parasympathetic activity and/or decreasing sympathetic activity, which may improve cardiac function and/or kidney function.

In one aspect, the disclosure is directed to a method comprising sensing a physiological parameter indicative of lung wetness within a patient, generating a neurostimulation signal configured to at least one of increase parasympathetic activity or decrease sympathetic activity within the patient to mitigate lung wetness based on the sensed physiological parameter, and delivering the neurostimulation signal to the patient.

In another aspect, the disclosure is directed to a system comprising a sensor that senses a physiological parameter indicative of lung wetness within a patient, a stimulation generator, and a processor. The processor controls the stimulation generator to generate and deliver a neurostimulation signal based on the sensed physiological parameter. The neurostimulation signal is configured to at least one of increase parasympathetic activity or decrease sympathetic activity within the patient to mitigate lung wetness.

In another aspect, the disclosure is directed to a system comprising means for sensing a physiological parameter indicative of lung wetness within a patient, means for generating a neurostimulation signal configured to at least one of increase parasympathetic activity or decrease sympathetic activity within the patient to mitigate lung wetness based on the sensed physiological parameter, and means for delivering the neurostimulation signal to the patient.

In another aspect, the disclosure is directed to a method of mitigating lung wetness of a lung of a patient. The method is characterized by implanting a medical device in the patient, the medical device comprising a sensor that senses a physiological parameter indicative of lung wetness within a patient, a stimulation generator, and a processor that controls the stimulation generator to generate and deliver a neurostimulation signal to the patient based on the sensed physiological parameter, wherein the neurostimulation signal is configured to at least one of increase parasympathetic activity or decrease sympathetic activity within the patient to mitigate lung wetness.

In another aspect, the disclosure is directed to a computer-readable medium comprising instructions. The instructions cause a processor to control a stimulation generator to generate and deliver a neurostimulation signal to a patient based on a sensed physiological parameter indicative of lung wetness, where the neurostimulation signal is configured to at least one of increase parasympathetic activity or decrease sympathetic activity within the patient to mitigate lung wetness.

In another aspect, the disclosure is directed to a computer-readable medium comprising instructions. The instructions cause a processor to perform any part of the techniques described herein. The instructions may be, for example, software instructions, such as those used to define a software or computer program. The computer-readable medium may be a computer-readable storage medium such as a storage device (e.g., a disk drive, or an optical drive), memory (e.g., a Flash memory, random access memory or RAM) or any other type of volatile or non-volatile memory that stores instructions (e.g., in the form of a computer program or other executable) to cause a programmable processor to perform the techniques described herein.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating an example therapy system that includes an implantable cardiac device (ICD) and an implantable neurostimulator (INS).

FIG. 2 is a conceptual diagram illustrating another example therapy system that includes an ICD and an INS.

FIG. 3 is a conceptual diagram illustrating the ICD and associated leads of the therapy systems of FIGS. 1 and 2 in greater detail.

FIG. 4 is a conceptual diagram illustrating another example ICD lead configuration.

FIG. 5 is a functional block diagram of an example ICD that generates and delivers electrical stimulation to a heart of a patient.

FIG. 6 is a functional block diagram of an example INS that generates and delivers electrical stimulation signals to a target tissue site other than cardiac tissue of a patient.

FIG. 7 is a functional block diagram of an example medical device programmer.

FIG. 8 is a flow diagram of an example technique for closed-loop delivery of neurostimulation to mitigate lung wetness.

FIG. 9 is a block diagram illustrating an example system that includes an external device, such as a server, and one or more computing devices that are coupled to the INS, ICD, and programmer shown in FIG. 1 via a network.

### DETAILED DESCRIPTION

In general, the disclosure is directed to delivering neurostimulation based on a sensed parameter indicative of lung wetness, e.g., a patient state or condition in which therapy delivery to mitigate fluid accumulation in a lung is desirable. Fluid accumulation in the lungs, also referred to as pulmonary edema or lung wetness, may be an indicator of a heart-related condition, such as heart failure or myocardial infarction, as well as an indicator of poor kidney function. More particularly, an elevated level of lung wetness, such as pulmonary edema or pleural effusion, may be an indicator of heart failure. In heart failure patients, the heart may be unable to pump enough blood from the heart, resulting in fluid accumulation in the lungs. In patients with poor kidney function, the kidney may insufficiently excrete fluid, which may result in an increase in fluid accumulation within the lungs of the patient. Neurostimulation therapy may help improve cardiac function and/or improve kidney function, which may mitigate lung wetness. Likewise, mitigating lung wetness may help reduce the need for neurostimulation therapy for reducing lung wetness. In this manner, neurostimulation and cardiac stimulation may complement each other. Delivering neurostimulation that mitigates lung wetness may reduce the occurrence of acute decompensated heart failure and hospitalization.

As described herein, neurostimulation therapy to a patient is configured to mitigate lung wetness by at least one of increasing parasympathetic activity and/or decreasing sympathetic activity. Increasing parasympathetic activity and/or decreasing sympathetic activity may help balance parasympathetic and sympathetic activity of the patient, e.g., balance the activity of the autonomic nervous system of patient 12, which may help improve the patient's lung wetness status (e.g., by decreasing the fluid accumulation in the lungs).

Neurostimulation therapy may help improve lung function, such as by preventing or reducing edema, water accumulation, and water retention in the lungs, causing the patient's body to more effectively excrete water in the lungs, and enabling the lungs to better respond to, mitigate, and/or effectively endure stresses within the lungs that are encountered during heart failure or poor kidney function. As one example, the lungs may become stressed when the heart is not able to properly manage blood flow and/or blood pressure in the lungs. Due to the stress, the lungs may work less effectively, which may result in excess lung wetness. Thus, stimulation of nerves associated with the lungs may help the lungs function more effectively in these situations.

Nerves associated with the lungs may directly or indirectly innervate the lungs. Spinal locations that associate directly with lung function and communication include, for example, nerves approximately within the region of the C7 and T1 through T3 vertebrae. Due to the interconnection of the entire spinal cord, delivering stimulation proximate to neural tissue near spinal vertebral sites that may not be directly associated with lung function and communication, such as approximately within the region of the T4 vertebra, may have similar therapeutic benefits as delivering stimulation at spinal cord locations more directly associated with lung function. Stimulating at a location directly or indirectly associated with lung function may result in an increase in endorphins and/or neurohormones that help the patient respond more effectively to stress and, thereby, may help mitigate lung wetness and other heart failure aspects. In some examples, a medical device may deliver stimulation approximately within the region of the T9 vertebra, which directly associates with the adrenal glands, to increase vitality and help mitigate lung wetness. As another example, a medical device may deliver stimulation at the approximate location of the kidneys of the patient, e.g., approximately within the region of the T9 through T12 vertebrae, which may help mitigate lung edema by enhancing body fluid regulation and/or drainage.

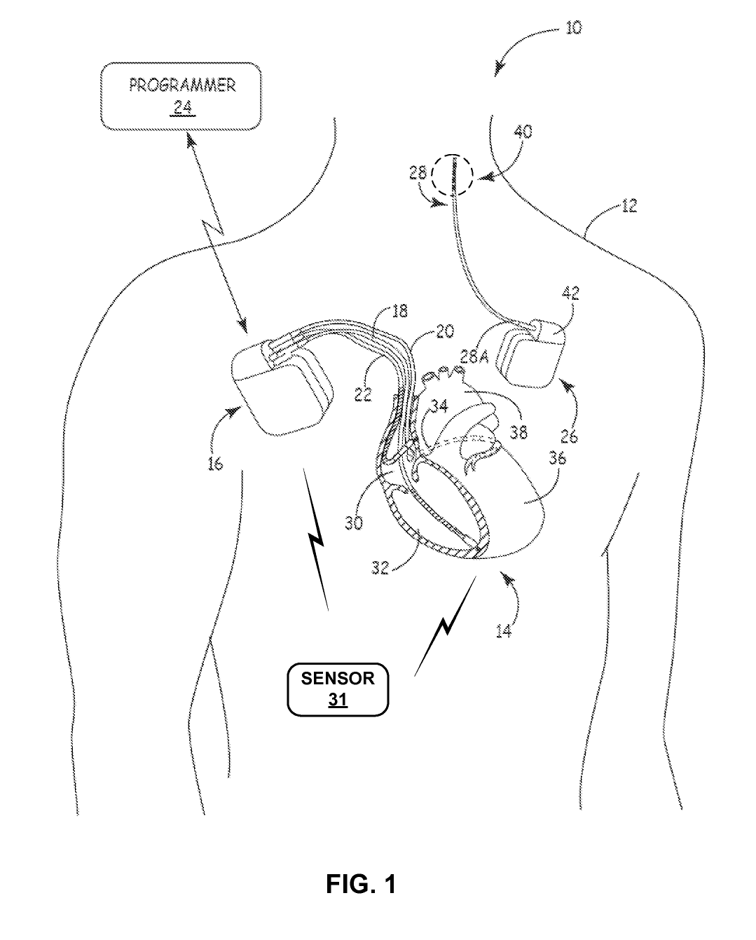

FIG. 1 is a conceptual diagram illustrating an example therapy system 10 that may be used to provide therapy to patient 12. Patient 12 ordinarily, but not necessarily, will be a human. Therapy system 10 includes implantable cardiac device (ICD) 16, which is connected (or “coupled”) to leads 18, 20, and 22, and programmer 24. ICD 16 may comprise, for example, an implantable pacemaker, cardioverter, and/or defibrillator that generates and delivers electrical signals to heart 14 of patient 12 via electrodes connected to one or more of leads 18, 20, and 22. In some examples, ICD 16 may deliver pacing pulses, but not cardioversion or defibrillation pulses, while in other examples, ICD 16 may deliver cardioversion or defibrillation pulses, but not pacing pulses. In addition, in further examples, ICD 16 may deliver pacing, cardioversion, and defibrillation pulses. ICD 16 may deliver any suitable type of pacing therapy, such as cardiac resynchronization therapy or pacing within the right ventricle or right atrium.

Therapy system 10 further comprises implantable neurostimulator (INS) 26, which is connected to lead 28. INS 26 may be any suitable implantable medical device (IMD) that includes a signal generator that generates electrical stimulation that may be delivered to a nerve or other tissue site of patient 12, e.g., proximate a vagus nerve, a spinal cord or heart 14 of patient 12. Example target tissue sites for electrical stimulation may include any suitable nonmyocardial tissue site or nonvascular cardiac tissue site. In some examples, INS 26 may deliver electrical stimulation that is delivered to peripheral nerves that innervate heart 14, or fat pads on heart 14 that may contain nerve bundles. In the example shown in FIG. 1, electrodes of lead 28 are positioned to deliver electrical stimulation to a vagus nerve (not shown) of patient 12. Although INS 26 is referred to throughout the remainder of the disclosure as a “neurostimulator” and as delivering neurostimulation pulses, in other examples, INS 26 may deliver electrical stimulation to any suitable tissue site within patient 12, which may or may not be proximate a nerve. For example, INS 26 may deliver stimulation signals to any suitable tissue site to modulate the activity of the autonomic nervous system of patient 12, e.g., parasympathetic, sympathetic and/or neurohormonal activity.

In some examples, the stimulation signals delivered by INS 26 may can utilized in conjunction and/or synergistically with pharmacological agents or other therapies to aid in modulating the activity of the autonomic nervous system of patient 12 and/or treating lung wetness. In some examples, INS 26 may include a reservoir to store a therapeutic agent and a pump and catheter to deliver the therapeutic agent to patient 12. In other examples, ICD 16 may deliver a therapeutic agent to patient 12, or patient 12 may receive a therapeutic agent via other means, e.g., orally or intravenously. Alternatively, an external or implantable medical device separate from ICD 16 and INS 26 can deliver a therapeutic agent to patient 12 to help manage lung wetness.

In the example shown in FIG. 1, ICD 16 and INS 26 are not physically connected to each other and each includes respective housings. Moreover, in the example shown in FIG. 1, ICD 16 is not mechanically connected to the electrodes of lead 28 and INS 26 is not mechanically connected to the electrodes of leads 18, 20, 22. Leads 18, 20, 22 that are coupled to ICD 16 extend into the heart 14 of patient 12 to sense electrical activity of heart 14 and/or deliver electrical stimulation to heart 14. In the example shown in FIG. 1, right ventricular (RV) lead 18 extends through one or more veins (not shown), the superior vena cava (not shown), and right atrium 30, and into right ventricle 32. Left ventricular (LV) coronary sinus lead 20 extends through one or more veins, the vena cava, right atrium 30, and into the coronary sinus 34 to a region adjacent to the free wall of left ventricle 36 of heart 14. Right atrial (RA) lead 22 extends through one or more veins and the vena cava, and into the right atrium 30 of heart 14. In other examples, ICD 16 may deliver stimulation therapy to heart 14 by delivering stimulation to an extravascular tissue site in addition to or instead of delivering stimulation via electrodes of intravascular leads 18, 20, 22.

ICD 16 may sense electrical signals attendant to the depolarization and repolarization of heart 14 via electrodes (not shown in FIG. 1) coupled to at least one of the leads 18, 20, 22. In some examples, ICD 16 provides pacing pulses to heart 14 based on the electrical signals sensed within heart 14. The configurations of electrodes used by ICD 16 for sensing and pacing may be unipolar or bipolar. ICD 16 may also provide defibrillation therapy and/or cardioversion therapy via electrodes located on at least one of the leads 18, 20, 22. ICD 16 may detect arrhythmia of heart 14, such as fibrillation of ventricles 32 and 36, and deliver defibrillation therapy to heart 14 in the form of electrical pulses. In some examples, ICD 16 may be programmed to deliver a progression of therapies, e.g., pulses with increasing energy levels, until a fibrillation of heart 14 is stopped. ICD 16 detects fibrillation employing one or more fibrillation detection techniques known in the art.

In the example of FIG. 1, INS 26 has been implanted in patient 12 proximate to a target stimulation site 40, such as a tissue site proximate a vagus nerve (not shown). For example, INS 26 may be subcutaneously or submuscularly implanted in the body of a patient 12 (e.g., in a chest cavity, lower back, lower abdomen, or buttocks of patient 12). INS 26 provides a programmable stimulation signal (e.g., in the form of electrical pulses or a continuous signal) that is delivered to target stimulation site 40 by implantable medical lead 28, and more particularly, via one or more stimulation electrodes carried by lead 28. INS 26 may also be referred to as a signal generator, stimulation generator or an electrical stimulator. In some examples, lead 28 may also carry one or more sense electrodes to permit INS 26 to sense electrical signals from target stimulation site 40. Furthermore, in some examples, INS 26 may be coupled to two or more leads, e.g., for bilateral or multi-lateral stimulation.

Proximal end 28A of lead 28 may be both electrically and mechanically coupled to connector 42 of INS 26 either directly or indirectly (e.g., via a lead extension). In particular, conductors disposed in the lead body may electrically connect stimulation electrodes (and sense electrodes, if present) of lead 28 to INS 26.

ICD 16 and/or INS 26 may sense thoracic impedance within patient 12 and/or other physiological parameters indicative of lung wetness via electrodes coupled to a respective at least one of leads 18, 20, 22, 28 and/or a housing of ICD 16 and/or INS 26. ICD 16 and/or INS 26 may also be in wired or wireless communication with other sensors implanted within patient 12, such as sensor 31 or external to patient 12. The sensors may be configured to sense one or more physiological parameters of patient 12 that indicate lung wetness. INS 26 may be configured to deliver neurostimulation therapy to patient 12 to help control the lung wetness of patient 12. In some examples, ICD 16 or INS 26, may, alone or in combination with each other or another sensor 31, detect (e.g., identify) a change in lung wetness based on the one or more sensed physiological parameters, such as thoracic impedance, posture, heart rate, respiration parameters (e.g., respiration rate, depth of inhalation and/or exhalation, rate profile of inhalation and/or exhalation, minute volume, and/or lung sounds), tissue impedance, activity level, blood or urine salinity, blood pressure, blood oxygen level, blood or urine pH, pleural effusion, edema in extremities or other non-pulmonary locations, and cardiac parameters (e.g., specific portions of an electrogram (EGM), such as the QRS or QRST segment, as described in further detail below).

ICD 16 and/or INS 26 may determine respiration parameters, such as the rate profile of inhalation and/or exhalation and the respiration rate, based on a determined thoracic impedance. Because shortness of breath or otherwise disturbed breathing may be characteristic of lung wetness, pulmonary edema, and pleural effusion, sensing respiration parameters may provide an indication of lung wetness status. In addition, lung sounds from the lungs, throat, or any other appropriate location within patient 12 may be detected using an acoustic sensor, such as a microphone. A characteristic of a signal generated by the acoustic sensor can be used to detect the lung sounds (e.g., cough, noisy breathing, raspy breathing or otherwise disturbed breathing) of interest. For example, an amplitude (e.g., mean, median, or peak amplitude) or a particular pattern in the time domain or frequency domain signal generated by the acoustic sensor can be associated with the occurrence of the disturbed breathing, and the threshold value or template for detecting the particular amplitude or pattern in the signal can be stored in a memory of ICD 16, INS 26, and/or another device. A processor of ICD 16, INS 26 or another device can compare a signal received from an acoustic sensor and compare the signal with the stored threshold value or template in order to determine whether patient 12 is exhibiting disturbed breathing that indicates pulmonary edema.

Noisy, raspy breathing, and/or excessive coughing may indicate lung wetness. A cough that worsens when the patient is sleeping or lying down may also indicate lung wetness. In order to determine whether the patient's cough worsens when patient 12 is lying down, ICD 16, INS 26 or another sensing module may monitor coughing in combination with time of day, e.g., via a clock, and/or patient posture, e.g., via a motion sensor that generates a signal indicative of patient posture (e.g., any one or more of a one-axis, two-axis or three-axis accelerometer, a gyroscope, a pressure transducer, or a piezoelectric crystal). The motion sensor can be used to determine when patient 12 is lying down and the clock can be used to determine when patient 12 is likely sleeping, although other techniques for detecting sleep (e.g., patient input or other physiological parameters) can also be used to determine when patient 12 is likely sleeping.

Changes in the frequency or intensity of cough (e.g., as indicated by the acoustic sensor alone or in combination with patient motion) when patient 12 is sleeping or lying down can indicate that the patient's cough worsens when patient 12 is lying down. Because respiration and coughing may produce characteristic motion, e.g., thoracic or body motion, ICD 16 and/or INS 26 may monitor respiration motion and/or cough motion using a motion sensor, which can be located within ICD 16, INS 26 or another external or implanted device. An intensity of a patient's cough can be indicated by a characteristic (e.g., an amplitude or a frequency domain characteristic) of a signal generated by an acoustic sensor when patient 12 coughs. In addition, in some examples, an intensity of a patient's cough can be indicated by a particular movement (e.g., as indicated by a signal generated by a motion sensor) associated with the cough (e.g., detected via a signal generated by an acoustic sensor or patient input indicating the occurrence of the cough).

Determining a patient's posture when patient 12 is likely sleeping may also provide an indication of lung wetness or congestion. Thus, in some examples, ICD 16, INS 26 or another sensor can determine a patient posture state and/or determine a patient sleep state. Some patients with lung congestion due to fluid accumulation in the lungs sleep in an upright position, e.g., by propping the upper torso up with pillows. The upright or near upright posture may allow patients experiencing lung congestion to breathe more comfortably. Therefore, a nighttime measurement of patient posture state may be indicative of lung wetness. Alternatively, the patient's posture may be sensed if one or more physiological parameters indicate that patient 12 is asleep or attempting to sleep. In some examples, upon detecting a physiological parameter indicative of lung wetness, ICD 16 and/or INS 26 can determine whether patient 12 is likely in a sleep state (e.g., based on a clock that indicates the time of day or based on patient input indicating patient 12 has entered a sleep state in which patient 12 is sleeping or attempting to sleep). ICD and/or INS 26 can then determine the patient posture state of patient 12 using any suitable technique, such as based on an accelerometer (e.g., one or more one-axis, two-axis or three-axis accelerometers). If patient 12 is in an upright posture state during the sleep state, ICD 16 and/or INS 26 can verify that the physiological parameter indicative of lung wetness is indicative of lung wetness.

ICD 16 and/or INS 26 may detect excess fluid around the lungs, referred to as pleural effusion, by sensing tissue impedance around the lungs. In addition to or instead of sensing tissue impedance to detect pleural effusion, ICD 16, INS 26 or another device configured to sense tissue conductivity or tissue perfusion proximate to the lungs may be used to detect pleural effusion. Pleural effusion may be associated with pulmonary edema and may result when fluid seeps from the lungs into the area surrounding the lungs. Thus, neurostimulation configured to mitigate lung wetness may also help mitigate pleural effusion. In some cases, edema in the patient's extremities, e.g., ankles, or other non-pulmonary locations, e.g., abdomen, may also indicate that there is also excess wetness in the lungs. ICD 16 and/or INS 26 may detect edema using remote sensors, e.g., in one or both of the ankles, or a general body sensor. Edema can be detected using any suitable technique, such as based on tissue conductivity or tissue perfusion.

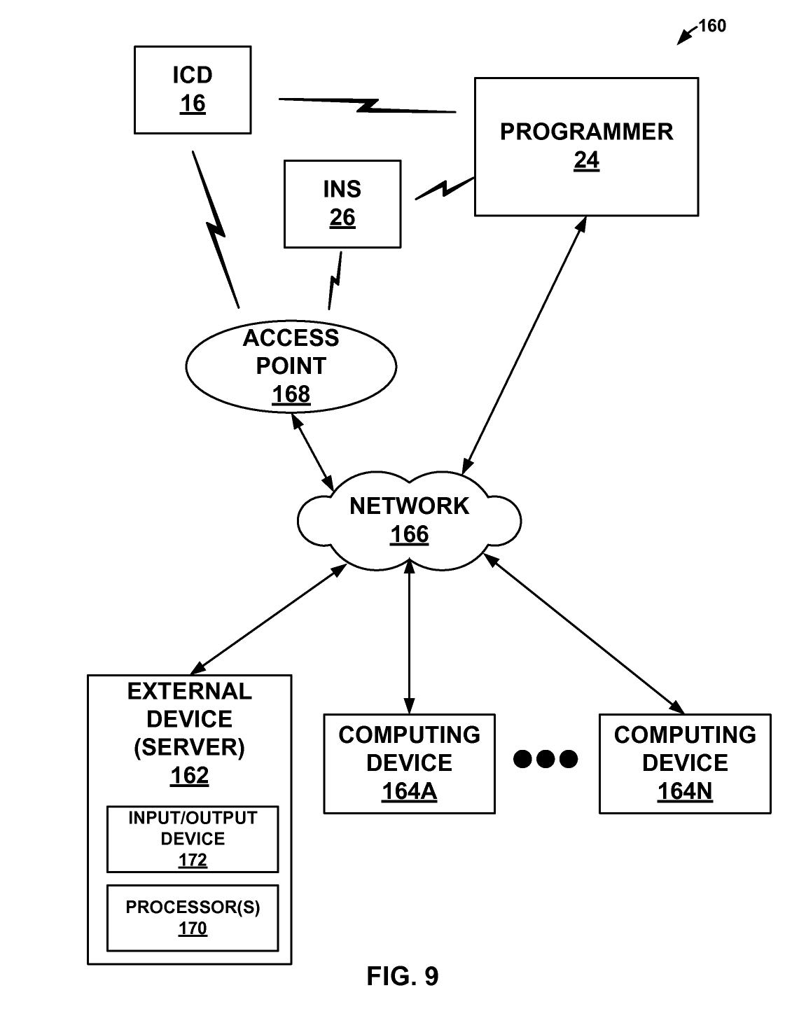

As another example, ICD 16, INS 26, and/or an external sensor may monitor patient weight and/or body fat percentage to determine a physiological parameter indicative of lung wetness. A patient's weight may indicate changes in fluid retention and, thus, pulmonary edema. The patient's weight may be communicated to ICD 16 and/or INS 26 based on a measurement taken by patient 12 and/or a healthcare provider. For example, the measurement may be manually inputted into an external device that communicates with ICD 16 and/or INS 26, such as programmer 24. ICD 16 and/or INS 26 may determine changes in the patient's weight based on the received measurements and adjust therapy delivery accordingly. As another example, an external sensor, e.g., a scale, may communicate with ICD 16 and/or INS 26, e.g., via a network (e.g., as shown in FIG. 9), such that ICD 16 and/or INS 26 automatically receives measurements. The patient's weight may be monitored on a periodic basis, e.g., daily.

Another physiological parameter indicative of lung wetness includes a body fat percentage of patient 12. ICD and/or INS 26 can determine the body fat percentage of patient 12 may be determined using any suitable technique. In one example, ICD 16 and/or INS 26 can receive information from an external sensor, e.g., a scale, as described with respect to measuring the patient's weight. As described with respect to measuring the patient's weight, the measurement values may be manually inputted into an external device that communicates with ICD 16 and/or INS 26, such as programmer 24, or automatically received from an external sensor that communicates with ICD 16 and/or INS 26, e.g., via a network (e.g., as shown in FIG. 9). One measurement of body fat percentage is bioelectrical impedance analysis (BIA). Some example external devices that monitor BIA include scales and handheld devices. These external devices may measure electrical parameters as signals pass through the fat, lean mass, and water in the body of patient 12. ICD 16, INS 26 or another device can apply an algorithm to determine body composition and total body water based on the BIA measurement.

In general, the more water a patient's body contains, the lower a percent body fat or BIA measurement will be. Thus, percent body fat or BIA may be utilized to help monitor edema progression, in general, which may generally correlate with cardiogenic pulmonary edema progression. Reductions in percent body fat measurements that occur in relatively short periods of time, e.g., within approximately 10 to approximately 24 hours, may indicate water retention and edema. Measurements of patient weight and/or body fat percentage may help support or verify lung wetness assessments made by other means of measuring lung wetness. In this way, the patient weight and body fat percentage can be secondary indicators of a condition in which lung wetness is present and it is desirable to minimize the lung wetness.

ICD 16, INS 26, and/or an external sensor may monitor one or more blood parameters of patient 12, such as one or more markers of renal function, levels of one or more electrolytes, levels of one or more liver enzymes, a concentration of brain naturetic peptide, and/or a blood sugar level. For example, ICD 16, INS 26, and/or an external sensor may monitor one or more biomarkers of renal function, such as creatinine and urea levels. In patients with poor kidney function, the kidneys may insufficiently excrete fluid, which may result in an increase in fluid accumulation within the lungs of the patient. Thus, biomarkers of renal function may be indicative of lung wetness. As another example, ICD 16, INS 26, and/or an external sensor may monitor one or more inflammatory markers, such a C-reactive protein. Inflammatory markers may indicate an inflammatory response that may result in inflammation of the bronchial passages and lung wetness.

As another example, ICD 16, INS 26, and/or an external sensor may monitor electrolytes, such as sodium and potassium to determine whether patient 12 is in a pulmonary edema state in which therapy delivery to patient 12 may be desirable. Sodium levels within patient 12 may be low if patient 12 is experiencing pulmonary edema. In some examples, patient 12 may receive diuretics to help reduce pulmonary edema, e.g., orally, intravenously, or via a reservoir, pump, and catheter of INS 26 or another external or implantable medical device. Diuretics may generally lower sodium and potassium levels. Levels of these electrolytes that increase subsequent to patient 12 receiving diuretics may be indicative of edema, including pulmonary edema. A clinician can specify the electrolyte levels that indicate the presence of pulmonary edema, and the therapy levels at which therapy delivery to patient 12 to help mitigate pulmonary edema may be desirable.

As another example of physiological parameters that can be indicative of pulmonary edema, ICD 16, INS 26, and/or an external sensor may monitor liver enzymes. Congestive hepatopathy is liver dysfunction that may be due to venous congestion that may generally result from cardiac dysfunction, such as congestive heart failure and pulmonary edema. High liver enzymes may result from passive congestion associated with cardiogenic pulmonary edema. Thus, elevated or ascending liver enzyme measurements may be utilized to help determine that pulmonary edema is present or worsening. Conversely, descending liver enzyme measurements may indicate the reversal of pulmonary edema. Therapy can be implemented in accordance with the presence/progression of the liver enzyme level.

As another example, ICD 16, INS 26, and/or an external sensor may monitor the concentration of brain naturetic peptide (BNP) in circulating blood. The concentration of BNP may increase with cardiogenic pulmonary edema.

As another example of physiological parameters that can be indicative of pulmonary edema, ICD 16, INS 26, and/or an external sensor may monitor a blood sugar level of patient 12. For example, ICD 16, INS 26, and/or an external sensor may monitor a blood sugar level of patient 12 if patient 12 has been diagnosed with diabetes. An indication of whether patient 12 is diabetic may be stored in a memory of ICD 16 and/or INS 26. A blood sugar level outside of a normal range, which may be stored in a memory of ICD 16 and/or INS 26, may result in an increase in lung wetness, e.g., in a diabetic patient due to the stress a blood sugar level outside of normal range may have on the body of patient 12. Conversely, stress on the body of patient 12 due to a blood sugar level outside of the normal range may degrade the body's metabolic management of lung wetness. ICD 16, INS 26, and/or an external sensor may monitor a blood sugar level of patient 12 in combination with one or more other parameters indicative of lung wetness. For example, ICD 16, INS 26, and/or an external sensor may monitor a primary parameter indicative of lung wetness to determine the need for neurostimulation configured to mitigate lung wetness and monitor a blood sugar level to adjust an intensity of therapy delivery. As another example, ICD 16, INS 26, and/or the external sensor can monitor the blood sugar level as a secondary indicator of pulmonary edema in order to confirm a determination that patient 12 is in a pulmonary edema state in which therapy delivery is desirable.

Blood parameters of patient 12 may be sensed by implantable blood analysis sensors that are integral to or communicate with ICD 16 and/or INS 26. As another example, blood tests may be performed by patient 12 or a health care provider by use of an externally located testing device. The results may be manually inputted into an external device that communicates with ICD 16 and/or INS 26. ICD 16 and/or INS 26 may automatically analyze the sensed blood parameter values and adjust or initiate neurostimulation therapy accordingly. For example, ICD 16 and/or INS 26 may compare the sensed blood parameter values to previous values to determine changes and control therapy. Alternatively, the external testing device may automatically communicate the results to ICD 16 and/or INS 26 to allow ICD 16 and/or INS 26 to analyze the sensed blood parameter values and adjust or initiate neurostimulation therapy accordingly.

In some examples, ICD 16, INS 26, and/or an external sensor may present patient weight, body fat percentage, and/or blood work parameters, such as electrolyte levels and blood sugar levels, to a clinician or other health care professional for analysis, e.g., via a network (e.g., as shown in FIG. 9) and/or programmer 24. This may allow a health care professional to analyze the sensed parameters and adjust therapy accordingly. In other example, INS 26 may deliver therapy based on a sensed parameter indicative of lung wetness, and the health care professional may adjust an intensity of the stimulation based on sensed measurements of patient weight, body fat percentage, and/or blood work parameters, such as electrolyte levels and blood sugar levels. As one example, the health care professional may adjust therapy via programmer 24 directly or indirectly (e.g., via a networked connection to programmer 24).

In some examples, ICD 16 and/or INS 26 includes a clock that indicates the time of day. Depending on the metabolism profile of patient 12, pulmonary edema may be worse in the evening while patient 12 is sleeping, in the morning, or at another time of day. Thus, in some examples, ICD 16 and/or INS 26 may initiate or adjust therapy for mitigating pulmonary edema based on the time of the day. As another example, ICD 16 and/or INS 26 may reduce an intensity, e.g., amplitude or duration, of stimulation at night if the intensity of stimulation delivered during the day becomes uncomfortable at night. ICD 16 and/or INS 26 may reduce therapy intensity according to the patient's typical sleep schedule, which can be input into programmer 24, ICD 16, INS 26, and/or automatically determined by ICD 16 or INS 26.

Physiological parameter values that are determined to be indicative of a lung wetness state for which therapy delivery to mitigate the lung wetness is desirable may vary based on the time of day. ICD 16 and/or INS 26 may monitor one or more sensed parameters in combination with the time of day in order to make the determination as to whether sensed parameters are indicative of lung wetness. During a patient's typical sleep schedule, the patient may be assumed to be lying down. A lying posture may influence the breathing pattern, lung sounds, and coughing patterns associated with lung wetness. ICD 16 and/or INS 26 may use the time of day in combination with these parameters to help account for posture-dependent changes in parameter values. Alternatively, a patient posture sensor (e.g., one or more one-axis, two-axis or three axis accelerometers, or one or more gyroscopes, or pressure transducers) may be utilized in combination with sensed parameters that vary with the patient's posture.

Additionally, it is believed that pulmonary edema may vary according to a chronobiological rhythm. Examples of known chronobiological rhythms include ultradian rhythms, such as heart rate, and circadian rhythms, such as the sleep-wake cycle. Chronobiologically assessing the sensed parameters may help achieve efficacious treatment and may also help time the therapy delivery to mitigate lung wetness. For example, ICD 16 and/or INS 26 may use sensed physiological parameter values and time of day to synchronize therapy delivery with the chronological rhythm of pulmonary edema.

ICD 16, INS 26, and/or an external sensor may also sense blood flow within the lungs of patient 12. Increased blood flow in the superior portions of the lungs, which may also be referred to as upper lobe diversion, may be indicative of cardiogenic pulmonary edema. Thus, an implanted or external sensor that measures blood flow to the superior portions of the lung may be utilized to initiate and/or adjust delivery of neurostimulation to mitigate lung wetness. ICD 16 and/or INS 26 may include and/or be wirelessly coupled to optical and/or ultrasonic sensors that monitor blood flow into the superior portions of the lungs.

INS 26 may deliver a neurostimulation signal to patient 12 in response to the determined change in lung wetness. The neurostimulation signal may be configured to at least one of increase parasympathetic activity or decrease sympathetic activity of patient 12. In some cases, increasing parasympathetic activity or decreasing sympathetic activity may modulate autonomic nervous activity of patient 12 in order to improve cardiac function of patient 12, which may help reduce lung wetness within patient 12. As one example, increasing parasympathetic activity or decreasing sympathetic activity may modulate autonomic nervous activity of patient 12 in order to decrease the heart rate of patient 12.

In some cases, increasing parasympathetic activity and/or decreasing sympathetic activity may modulate neurohormonal activity of patient 12 to prevent or reverse the progression of heart failure of patient 12, which may help reduce lung wetness within patient 12. For example, increasing parasympathetic activity and/or decreasing sympathetic activity may at least partially prevent or reverse activation of the sympathetic nervous and/or renin-angiotensin-aldosterone systems, which have been attributed to the progression of heart failure. As another example, increasing parasympathetic activity and/or decreasing sympathetic activity may regulate intracardiac paracrine hormone levels and/or whole body hormone levels, e.g., via the central nervous system. In this manner, INS 26 may deliver a neurostimulation signal configured to at least one of increase parasympathetic activity or decrease sympathetic activity of patient to regulate neurohormonal activity within patient 12.

ICD 16 or INS 26 may, alone or in combination with each other or other sensing devices, sense the response of patient 12 to the delivered neurostimulation signal via electrodes coupled to at least one of the respective leads 18, 20, 22, 28 and/or other sensors in wired and/or wireless communication with ICD 16 and/or INS 26. Examples of physiological parameters of patient 12 that may be used to detect the patient response to the neurostimulation therapy may include heart contractility, lung wetness, respiration rate, heart rate, heart rate variability, blood pressure, bladder size, bladder functional activities, urine output, lung function, lung composition, and/or nerve activity. The detected patient response to the neurostimulation configured to increase parasympathetic activity or decrease sympathetic activity may indicate whether patient 12 has responded to the neurostimulation, e.g., whether the lung wetness of patient 12 has decreased, whether heart 14 of patient 12 has improved its mechanical function (e.g., as indicated by heart contractility) or improved stroke volume, or otherwise improved cardiac function.

In the example shown in FIG. 1, INS 26 provides electrical stimulation therapy of a parasympathetic nerve, such as a vagus nerve, of patient 12 to increase parasympathetic activity. Stimulation of a parasympathetic nerve of patient 12 may help mitigate lung wetness. In contrast to therapy systems that merely increase sympathetic activity and increase the heart rate of patient 12 to provide an acute decrease in lung wetness, increasing parasympathetic activity may help improve physiological functions of patient 12 that help mitigate lung wetness over a longer period of time (e.g., resulting in longer lasting effects). Therefore, delivering stimulation to increase parasympathetic activity may provide longer lasting therapeutic effects compared to systems that merely increase sympathetic activity. For example, it is believed that stimulation of a parasympathetic nerve may cause vasodilation, e.g., of the vessels in the lungs and/or heart 14 of patient 12. It is further believed that vasodilation may help mitigate lung wetness by, for example, reducing arterial blood pressure and, hence, cardiac afterload. The reduced afterload may increase cardiac function. The increased cardiac function may result in increased cardiac output and stroke volume. The increased cardiac output and stroke volume may aid in removing blood from the lungs.

Stimulation of a parasympathetic nerve and/or vasodilation that results from such neurostimulation may also improve renal function. Increased renal function may result in increased urine output and decreased water retention. As a result of reduced water retention, blood volume may decrease and result in a reduction in cardiac preload. The reduced preload may increase cardiac function. The increased cardiac function may result in increased cardiac output and stroke volume. The increased cardiac output and stroke volume may aid in removing blood from the lungs.

Stimulation of a parasympathetic nerve and/or vasodilation that results from such neurostimulation may also directly improve heart function. For example, stroke volume and/or cardiac output may increase subsequent to such neurostimulation. The increased cardiac output and stroke volume may aid in removing blood from the lungs.

Stimulation of a parasympathetic nerve of patient 12 may, additionally or alternatively, alter the fluid permeability and inflammatory response of the bronchial passages, blood vessels, and lymphatic system within the lungs and/or reduce fluid generation in the lungs. These responses may help reduce the burden on the heart and may also help the lungs more effectively dispose of fluid in the lungs.

Therapeutic systems that merely increase sympathetic activity in order to mitigate lung wetness provide a temporary decrease in lung wetness by increasing heart rate and increasing the amount of fluid that is removed from the lungs of the patient by increasing the heart rate. In contrast, as discussed above, delivering stimulation to increase parasympathetic activity may provide longer lasting therapeutic effects compared to systems that merely increase sympathetic activity by improving cardiac contractility, which may help increase the volume of fluid that is moved out of the patient's lungs per cardiac contraction.

INS 26 may also provide stimulation signals (e.g., to a parasympathetic nerve) to help slow intrinsic rhythms of heart 14, e.g., by increasing parasympathetic activity and/or decreasing sympathetic activity, which may complement cardiac rhythm management therapy (e.g., antitachycardia pacing, cardioversion or defibrillation) delivered by ICD 16. For example, stimulation of a parasympathetic nerve of patient 12 may help reduce the incidence of tachyarrhythmia of heart 14. Additionally, improving cardiac function may result in a decrease in lung wetness. Decreasing lung wetness may also improve cardiac function, e.g., decrease symptoms of heart failure. Therefore, decreasing lung wetness may reduce the need for cardiac therapy from ICD 16 and/or compliment cardiac therapy delivered by ICD 16.

INS 26 may sense one or more parameters indicative of lung wetness, such as thoracic impedance, within patient 12 via electrodes coupled to lead 28. INS 26 may also be in wired or wireless communication with other sensors implanted within patient 12 for detection of lung wetness or other physiological parameters. INS 26, alone or in combination with ICD 16, may determine a change in lung wetness based on the one or more sensed physiological parameters and deliver a neurostimulation signal to patient 12 in response to the determined change in lung wetness. The neurostimulation signal may be configured to at least one of increase parasympathetic activity or decrease sympathetic activity. In some examples, ICD 16 may determine the change in lung wetness and alert INS 26 of the change. INS 26 and/or ICD 16 may sense the response of patient 12 to the neurostimulation signal via electrodes and/or other sensors in wired and/or wireless communication with INS 26 and/or ICD 16, respectively. In some examples, INS 26 may also sense parameters that ICD 16 may utilize to initiate and/or adjust delivery of cardiac therapy.

In other examples, electrodes of lead 28 may be positioned to deliver electrical stimulation to any other suitable nerve, organ, muscle or muscle group in patient 12, to increase parasympathetic activity and/or decrease sympathetic activity in order to mitigate lung wetness of patient 12. In some examples, INS 26 may deliver electrical stimulation to other sympathetic or parasympathetic nerves, baroreceptors, or the carotid sinus or a cardiac branch of the vagal trunk of patient 12 in order to mitigate lung wetness and/or complement the delivery of therapy by ICD 16. As examples, INS 26 may deliver electrical stimulation to the median nerve and/or one or more baroreceptors of patient 12 to increase parasympathetic activity and/or decrease sympathetic activity to mitigate lung wetness.

INS 26 may deliver electrical stimulation with therapy parameters values that may be configured to mitigate lung wetness by increasing parasympathetic activity and/or decreasing sympathetic activity. The therapy parameters for INS 26 may include an electrode combination, and an amplitude, which may be a slew rate, a current or voltage amplitude, a duty cycle, and, if INS 26 delivers electrical pulses, a pulse width, and a pulse rate for stimulation signals to be delivered to patient 12. An electrode combination may include a selected subset of one or more electrodes located on implantable lead 28 coupled to INS 26. By selecting particular electrode combinations, a clinician may target particular anatomic structures within patient 12, such as particular portions of a nerve of patient 12. In addition, by selecting values for amplitude, pulse width, and pulse rate, the physician can attempt to generate an efficacious therapy for patient 12 that is delivered via the selected electrode subset.

To mitigate lung wetness, INS 26 may generate and deliver electrical stimulation signals that are configured to mitigate lung wetness by increasing parasympathetic activity and/or decreasing sympathetic activity. Examples of such electrical stimulation signals include electrical stimulation signals having a relatively high frequency stimulation, e.g., at a frequency between approximately 10 Hertz (Hz) and approximately 1 kilohertz (kHz). If INS 26 delivers electrical pulses, INS 26 may, as one example, deliver pulses with a pulse width of approximately 0.2 milliseconds. Additionally, INS 26 may deliver continuous therapy, e.g., for a duration of approximately 2 hours. As another example, INS 26 may deliver stimulation in intervals, such as repeating 10 second intervals. As yet another example, INS 26 may synchronize stimulation delivery with the cardiac cycle of heart 14, e.g., such that INS 26 delivers stimulation pulses synchronized with the R-wave of the cardiac cycle. Additionally or alternatively, other therapy parameter values may be selected to increase parasympathetic activity and/or decrease sympathetic activity. Other stimulation parameter values are contemplated and may, for example, depend upon the particular patient 12 or the nerve that is stimulated to increase parasympathetic activity or decrease sympathetic activity.

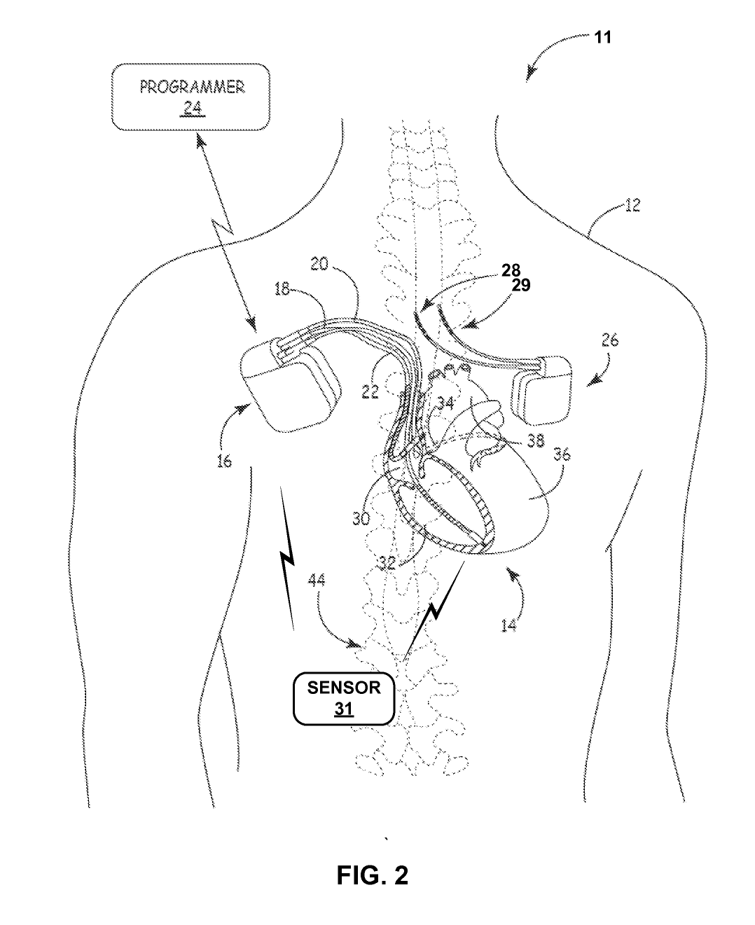

As another example, as shown in FIG. 2, INS 26 delivers electrical stimulation to spinal cord 44 of patient 12 in order to increase parasympathetic activity or decrease sympathetic activity, which may help mitigate lung wetness of patient 12. As previously described, INS 26 may deliver stimulation using therapy parameter values selected to increase parasympathetic activity and/or decrease sympathetic activity. When INS 26 delivers stimulation to spinal cord 44, INS 26 may deliver stimulation using a signal that varies over time, e.g., from high amplitude to low amplitude, low amplitude to high amplitude, high frequency to low frequency, or low frequency to high frequency. In some examples, the stimulation signal varies according to a predetermined pattern, e.g., that repeats over time. The stimulation signal may cycle between parameters configured to increase parasympathetic activity and parameters configured to decrease sympathetic autonomic activity, e.g., according to a predetermined pattern. For example, INS 26 can alternate between delivering a first stimulation signal that increases parasympathetic activity of patient 12 and a second stimulation signal that decreases sympathetic activity of patient 12, where the first and stimulation signals are generated with respective sets of stimulation parameters having at least one different parameter value. In some cases, alternations may provide particularly efficacious therapy. As one example, the alternation may be a reversal of the polarity of the applied stimulation.

As one example, INS 26 may deliver stimulation at a frequency of approximately 20 Hz with a pulse duration of approximately 0.5 millisecond (ms) at an amplitude of approximately 5 Volts (V). INS 26 may cycle between periods of delivering the stimulation signal and periods of not delivering the stimulation signal. For example, INS 26 may deliver the stimulation signal for approximately 10 seconds at a time with a period of approximately 50 seconds between each of the periods of stimulation. In some examples, INS 26 may deliver stimulation with a frequency of approximately 5 Hz to approximately 100 Hz, a pulse duration of approximately 0.1 ms to approximately 1.0 ms, and an amplitude of approximately 1 V to approximately 10 V. INS 26 may deliver the stimulation signal continuously or may cycle between periods of delivering the stimulation signal and periods of not delivering the stimulation signal. As one example, INS 26 may deliver the stimulation signal for approximately 0.1 seconds to approximately 100 seconds at a time with a period of approximately 0.1 seconds to approximately 100 seconds between each of the periods of stimulation.

In the example shown in FIG. 2, INS 26 is coupled to two leads 28, 29, which may facilitate bilateral spinal cord stimulation of patient 12. In other examples, INS 26 may be coupled to a single lead 28 or 29 or more than two leads. Although leads 28, 29 are shown to be introduced into the epidural space of spinal cord 44 via the thoracic column in the example shown in FIG. 2, in other examples, leads 28, 29 may be introduced into the epidural space of spinal cord 44 near the cervical or lumbar regions. Electrodes of leads 28, 29 may be positioned within an intrathecal space or epidural space of spinal cord 44, or, in some examples, adjacent nerves that branch off of spinal cord 44. Stimulation of spinal cord 44 or nerves branching therefrom by INS 26 may help mitigate lung wetness.

INS 26 may stimulate spinal cord 44 or nerves branching therefrom to increase parasympathetic activity and/or decrease sympathetic activity within patient 12 to mitigate lung wetness. INS 26 may stimulate spinal cord 44 and/or nerves extending from spinal cord 44 at the approximate location of the heart and/or lungs of patient 12, e.g., approximately within the region of the T1-T6 vertebrae. In some examples, INS 26 may stimulate proximate to spinal cord 44 at one or more locations directly associated with lung function and communication, such as approximately in the region of the C7 and T1 through T3 vertebrae. It is believed that the stimulation of spinal cord 44 and/or nerves extending from spinal cord 44 at the approximate location of the heart and/or lungs of patient 12 may dilate the veins that carry blood from the lungs to the heart, which may decrease the fluid pressure of the blood entering the lungs. It is also believed that the stimulation may dilate peripheral vessels, which may assist in decreasing the pressure load on portions of heart 14, such as the left ventricle.

INS 26 may, additionally or alternatively, stimulate proximate to other spinal vertebral sites, such as neural tissue proximate to the T4 vertebra. Delivering stimulation proximate to neural tissue proximate spinal vertebral sites that may not be directly associated with lung function and communication may have similar therapeutic benefits due to the communication interconnect of the entire spinal cord 44. INS 26 may indirectly stimulate lung function, thereby reducing pulmonary edema, by stimulating at such spinal vertebral sites. In some cases, positioning electrodes proximate to spinal vertebral sites indirectly associated with lung function may be less invasive or more convenient to reach with an implantable medical device or lead compared to positioning electrodes proximate to spinal vertebral sites directly associated with lung function. Stimulating at a location directly or indirectly associated with lung function may result in an increase in endorphins and/or neurohormones that help patient 12 respond more effectively to stress and, thereby, may help mitigate lung wetness and other heart failure aspects. In some examples, INS 26 may deliver stimulation approximately within the region of the T9 vertebra, which directly associates with the adrenal glands, to increase vitality and help mitigate lung wetness.

In some examples, INS 26 may stimulate the central nervous system. As described previously, INS 26 may stimulate spinal cord 44 of the central nervous system, e.g., in the cervical, thoracic, and/or lumbar regions. INS 26 may stimulate preganglionic neural tissue of the central nervous system proximate to spinal cord 44. Stimulating preganglionic neural tissue may allow INS 26 to deliver lower intensity stimulation, e.g., lower amplitude and/or lower frequency, compared to stimulating tissue of the peripheral nervous system. This may be useful for conserving the power source of INS 26 without adversely affecting the therapeutic benefits to patient 12. Additionally, preganglionic tissue may be more convenient to access than peripheral neural tissue. For example, implanting leads 28 and 29 proximate to preganglionic tissue may be less invasive than implanting leads 28 and proximate to peripheral neural tissue.

Additionally or alternatively, INS 26 may deliver electrical stimulation to a target tissue site within the brain of patient 12, e.g., at one or more centers that regulate autonomic activity. Example target tissue sites within the brain of patient 12 include, but are not limited to, the dorsal vagal motonucleus, nucleus ambiguus, nucleus tractus solitarii, hypothalamus, and/or spinal intermediolateral column. In such examples, one or more of leads 28 and 29 may be implanted within the brain of patient 12. Delivering stimulation to a target tissue site within the brain may affect the parasympathetic and sympathetic activity in a similar manner to that discussed above with respect to stimulation delivered to tissue sites proximate to spinal cord 44. Thus, any of the techniques described herein can be applied to delivering stimulation to the brain of patient 12 to help manage pulmonary edema.

In other examples, INS 26 may stimulate one or more ganglion proximate to spinal cord 44. Stimulating one or more ganglion may allow INS 26 to deliver lower intensity stimulation, e.g., lower amplitude and/or lower frequency, than stimulating tissue of the peripheral nervous system further away from spinal cord 44. Additionally, ganglia may be more convenient to access than peripheral neural tissue further away from spinal cord 44. For example, implanting leads 28 and 29 proximate to one or more ganglion may be less invasive than implanting leads 28 and proximate to other peripheral neural tissue further away from spinal cord 44.

As an alternative, INS 26 may deliver stimulation at the approximate location of the kidneys of patient 12, e.g., approximately within the region of the T9 through T12 vertebrae. INS 26 may mitigate lung wetness by decreasing, e.g., inhibiting, renal sympathetic activity and/or increasing renal parasympathetic activity to increase fluid excretion by the kidneys of patient 12. An increase in fluid excretion by the kidneys may in turn decrease fluid accumulation within the lungs of patient 12. Bladder size may be indicative of fluid retention by the kidneys and renal sympathetic tone. Accordingly, bladder size may be monitored, e.g., using a strain gauge or other pressure sensor proximate to a wall of the bladder of patient 12, to provide an indication of renal sympathetic activity. A strain gauge may be positioned to detect changes in the mechanical deformation of the patient's bladder, which may indicate the relative bladder size and relative changes in the bladder size. The strain gauge may generate an electrical signal that changes as a function of the mechanical deformation of the patient's bladder and transmit the signal to INS 26, e.g., via wireless communication or a wired link.

A pressure sensor may also generate a signal that changes as a function of the bladder size. In particular, the therapy system may include a pressure sensor that senses changes in pressure in the bladder wall of the patient. Examples of strain gauges and pressure sensors that may be used to monitor bladder size are described in common-assigned U.S. patent application Ser. No. 11/414,527 to Rondoni et al., which is entitled, “TREE-BASED ELECTRICAL STIMULATOR PROGRAMMING” and was filed on Apr. 28, 2006, the entire content of which is incorporated herein by reference.

Bladder size is described as one example parameter indicative of fluid retention by the kidneys and renal sympathetic tone. Other parameters, such as functional activities of the bladder and urine flow, may also provide indications of fluid retention by the kidneys and renal sympathetic tone. A sensor, e.g., proximate to the bladder of patient 12, may monitor functional activities of the bladder and/or urine flow, in addition to or as an alternative to bladder size.

Stimulation of spinal cord 44 or nerves branching therefrom by INS 26 may also help prevent or mitigate occurrences of tachyarrhythmias and may reduce the level of aggressiveness of the cardiac therapy, such as pacing, cardioversion or defibrillation, delivered by ICD 16. In this way, ICD 16 and INS 26 may operate in conjunction with each other to help prevent arrhythmias of heart 14 of patient 12, as well as to terminate detected arrhythmias.

In some examples, programmer 24 may be a handheld computing device or a computer workstation. Programmer 24 may include a user interface that receives input from a user. The user interface may include, for example, a keypad and a display, which may for example, be a cathode ray tube (CRT) display, a liquid crystal display (LCD) or light emitting diode (LED) display. The keypad may take the form of an alphanumeric keypad or a reduced set of keys associated with particular functions. Programmer 24 can additionally or alternatively include a peripheral pointing device, such as a mouse, via which a user may interact with the user interface. In some examples, a display of programmer 24 may include a touch screen display, and a user may interact with programmer 24 via the display.

A user, such as a physician, technician, patient or other clinician, may interact with programmer 24 to communicate with ICD 16 and/or INS 26. For example, the user may interact with programmer 24 to retrieve physiological or diagnostic information from ICD 16 and/or INS 26. A user may also interact with programmer 24 to program ICD 16 and INS 26, e.g., select values for operational parameters of ICD 16 and INS 26, respectively.

For example, the user may use programmer 24 to retrieve information from ICD 16 regarding the rhythm of heart 14, trends therein over time, or tachyarrhythmia episodes. As another example, the user may use programmer 24 to retrieve information from ICD 16 regarding other sensed physiological parameters of patient 12, such as electrical depolarization/repolarization signals from the heart (referred to as an EGM), intracardiac or intravascular pressure, activity, posture, respiration, or thoracic impedance. As another example, the user may use programmer 24 to retrieve information from ICD 16 regarding the performance or integrity of ICD 16 or other components of system 10, such as leads 18, 20, and 22, or a power source of ICD 16.

The user may use programmer 24 to program a therapy progression, select electrodes used to deliver defibrillation pulses, select waveforms for the defibrillation pulse, or select or configure a fibrillation detection algorithm for ICD 16. The user may also use programmer 24 to program aspects of other therapies provided by ICD 16, such as cardioversion or pacing therapies. For example, with the aid of programmer 24, a user may select therapy parameters for ICD 16. The therapy parameters may include an electrode combination, a current or voltage amplitude, a pulse width, and a pulse rate for stimulation signals to be delivered to patient 12. An electrode combination may include a selected subset of one or more electrodes located on implantable leads 18, 20, 22 that are coupled to ICD 16. The electrode combination may also refer to the polarities of the electrodes in the selected subset. By selecting values for amplitude, pulse width, and pulse rate, the physician can attempt to generate an efficacious therapy for patient 12 that is delivered via the selected electrode subset.

In some examples, the user may activate certain features of ICD 16 by entering a single command via programmer 24, such as depression of a single key or combination of keys of a keypad or a single point-and-select action with a pointing device.

As another example, the user may use programmer 24 to retrieve information from INS 26 regarding the performance or integrity of INS 26 or leads 28, 29 (if INS 26 is connected to more than one lead) or a power source of INS 26. With the aid of programmer 24 or another computing device, a user may select values for therapy parameters for controlling therapy delivery by INS 26. The values for the therapy parameters may be organized into a group of parameter values referred to as a “therapy program” or “therapy parameter set.” “Therapy program” and “therapy parameter set” are used interchangeably herein.

In the case of electrical stimulation, the therapy parameters for INS 26 may include an electrode combination, and an amplitude, which may be a current or voltage amplitude, and, if INS 26 delivers electrical pulses, a pulse width, and a pulse rate for stimulation signals to be delivered to patient 12. An electrode combination may include a selected subset of one or more electrodes located on implantable lead 28 coupled to INS 26. By selecting particular electrode combinations, a clinician may target particular anatomic structures within patient 12. In addition, by selecting values for amplitude, pulse width, and pulse rate, the physician can attempt to generate an efficacious therapy for patient 12 that is delivered via the selected electrode subset.

Programmer 24 may communicate with ICD 16 and INS 26 via wireless communication using any techniques known in the art. Examples of communication techniques may include, for example, low frequency or radiofrequency (RF) telemetry, but other techniques are also contemplated. In some examples, programmer 24 may include a programming head that may be placed proximate to the patient's body near the ICD 16 and INS 26 implant sites in order to improve the quality or security of communication between ICD 16 or INS 26, respectively, and programmer 24.

In other examples of therapy systems 10 (FIG. 1), 11 (FIG. 2), ICD 16 may be configured to deliver cardiac rhythm therapy to a nonmyocardial tissue site. For example, ICD 16 may be a subcutaneous pacemaker, cardioverter, and/or defibrillator that delivers at least one of pacing, cardioversion or defibrillation therapy to heart 14 via two or more extravascular electrodes, and, in some cases, without intravascular electrodes. Examples of extravascular electrodes include, but are not limited to, subcutaneous coil electrodes, which may be positioned within a subcutaneous tissue layer of patient 12, subcutaneous ring electrodes, subcutaneous plate electrodes, subcutaneous patch or pad electrodes, or any other type of extrathoracic electrode, such as a submuscular electrode, an epicardial electrode or an intramural electrode.

In addition, in other examples of therapy systems 10 (FIG. 1), 11 (FIG. 2), ICD 16 may not necessarily be configured to deliver cardiac rhythm therapy to heart 14 of patient 12. In some examples, ICD 16 may provide cardiac monitoring of heart 14, e.g., to monitor an electrical cardiac signal (e.g., an electrogram or electrocardiogram) of patient 12, intrathoracic impedance, heart rate, blood oxygen saturation, and other physiological parameters that may be indicative of cardiac function of patient 12.

Although both ICD 16 and INS 26 are illustrated in the example of FIG. 1, therapy system 10 does not necessarily include ICD 16. In other examples, a therapy system may include INS 26 but not ICD 16. For example, INS 26 may sense one or more physiological parameters of patient 12 that are indicative of lung wetness, e.g., as described above, and control the delivery of neurostimulation to patient 12 based on the sensed parameter indicative of lung wetness. In this way, INS 26 may perform any of the techniques described herein for mitigating lung wetness.

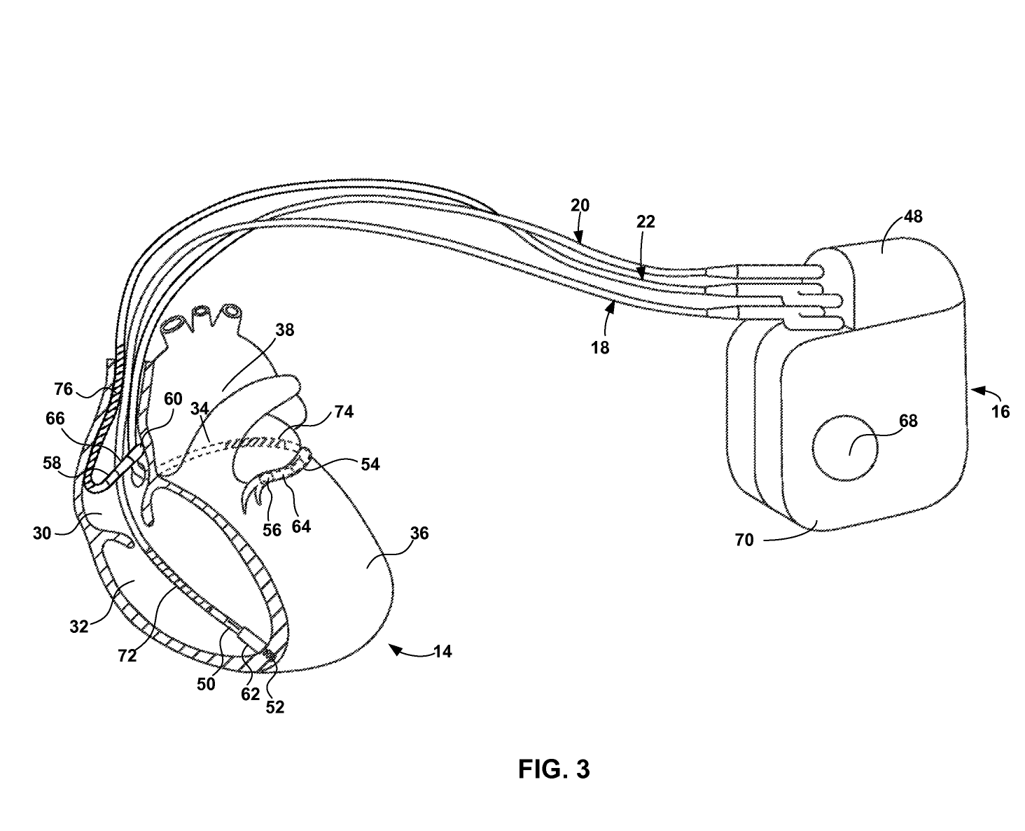

FIG. 3 is a conceptual diagram illustrating ICD 16 and leads 18, 20, 22 of therapy system 10 in greater detail. Leads 18, 20, 22 may be electrically coupled to a signal generator, a sensing module, or other modules of ICD 16 via connector block 48. In some examples, proximal ends of leads 18, 20, 22 may include electrical contacts that electrically couple to respective electrical contacts within connector block 48. In addition, in some examples, leads 18, 20, 22 may be mechanically coupled to connector block 48 with the aid of set screws, connection pins or another suitable mechanical coupling mechanism.

Each of the leads 18, 20, 22 includes an elongated insulative lead body, which may carry a number of concentric coiled conductors separated from one another by tubular insulative sheaths. Other lead configurations are also contemplated, such as lead configurations in which at least some of the conductors of the leads 18, 20, 22 are not coiled. In the illustrated example, electrodes 50 and 52 are located proximate to a distal end of lead 18. In addition, electrodes 54 and 56 are located proximate to a distal end of lead 20 and electrodes 58 and 60 are located proximate to a distal end of lead 22.

Electrodes 50, 54 and 58 may take the form of ring electrodes, and electrodes 52, 56 and 60 may take the form of extendable helix tip electrodes mounted retractably within insulative electrode heads 62, 64, and 66, respectively. Each of the electrodes 50, 52, 54, 56, 58, and 60 may be electrically coupled to a respective one of the coiled conductors within the lead body of its associated lead 18, 20, 22, and thereby coupled to respective ones of the electrical contacts on the proximal end of leads 18, 20 and 22.

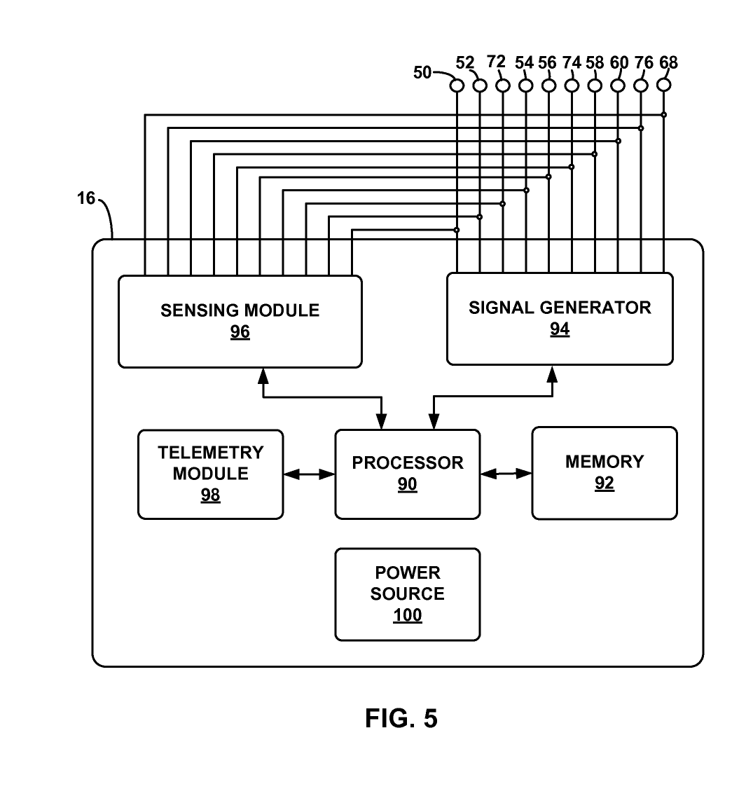

Electrodes 50, 52, 54, 56, 58, and 60 may sense electrical signals attendant to the depolarization and repolarization of heart 14. The electrical signals are conducted to ICD 16 via the respective leads 18, 20, 22. In some examples, ICD 16 also delivers pacing pulses via electrodes 50, 52, 54, 56, 58, and 60 to cause depolarization of cardiac tissue of heart 14. In some examples, as illustrated in FIG. 2, ICD 16 includes one or more housing electrodes, such as housing electrode 68, which may be formed integrally with an outer surface of hermetically-sealed housing 70 of ICD 16 or otherwise coupled to housing 70. In some examples, housing electrode 68 is defined by an uninsulated portion of an outward facing portion of housing 70 of ICD 16. Other division between insulated and uninsulated portions of housing 70 may be employed to define two or more housing electrodes. In some examples, housing electrode 68 comprises substantially all of housing 70. Any of the electrodes 50, 52, 54, 56, 58, and 60 may be used for unipolar sensing or pacing in combination with housing electrode 68. As described in further detail with reference to FIG. 5, housing 70 may enclose a signal generator that generates cardiac pacing pulses and defibrillation or cardioversion shocks, as well as a sensing module for monitoring the patient's heart rhythm or other physiological parameters.

Leads 18, 20, 22 also include elongated electrodes 72, 74, 76, respectively, which each may take the form of a coil. ICD 16 may deliver defibrillation pulses to heart 14 via any combination of elongated electrodes 72, 74, 76, and housing electrode 68. Electrodes 68, 72, 74, 76 may also be used to deliver cardioversion pulses to heart 14. Electrodes 72, 74, 76 may be fabricated from any suitable electrically conductive material, such as, but not limited to, platinum, platinum alloy or other materials known to be usable in implantable defibrillation electrodes.

The configuration of therapy system 10 illustrated in FIGS. 1-3 are merely examples. In other examples, a therapy system may include epicardial leads and/or patch electrodes instead of or in addition to the transvenous leads 18, 20, 22 illustrated in FIG. 1. Further, ICD 16 and INS 26 need not be implanted within patient 12. In examples in which ICD 16 is not implanted in patient 12, ICD 16 may deliver pacing pulses and other therapies to heart 14 via percutaneous leads that extend through the skin of patient 12 to a variety of positions within or outside of heart 14. In examples in which INS 26 is not implanted in patient 12, INS 26 may deliver electrical stimulation to target tissue sites within patient 12 via external electrodes or via percutaneous leads that extend through the skin of patient 12.

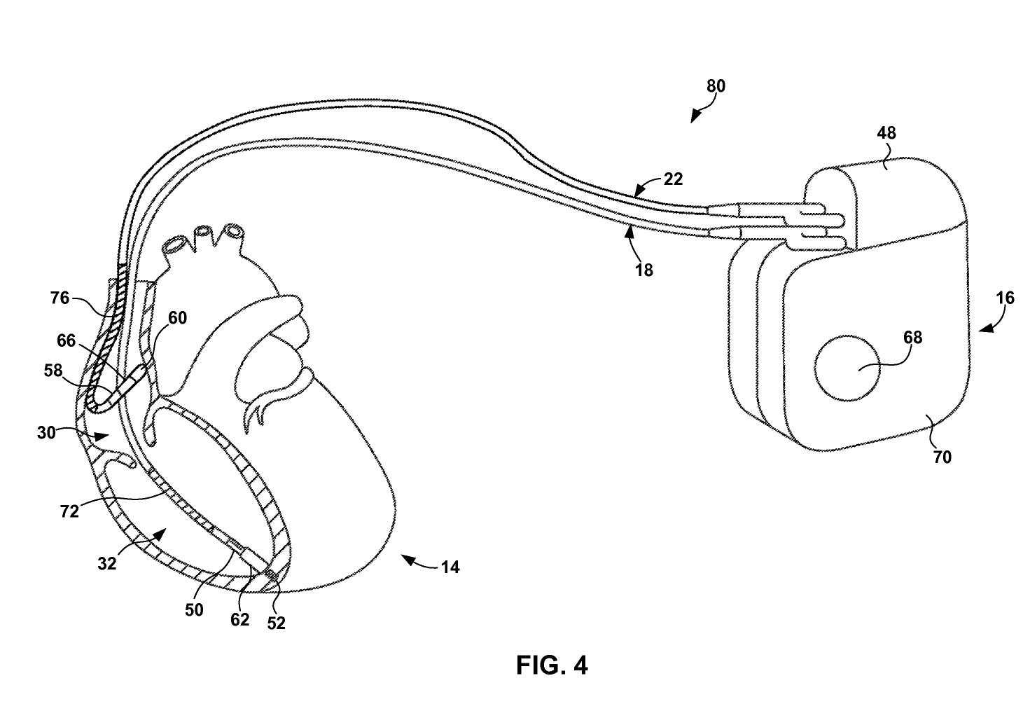

In other examples of therapy systems that provide electrical stimulation therapy to heart 14, a therapy system may include any suitable number of leads coupled to ICD 16, and each of the leads may extend to any location within or proximate to heart 14. For example, other examples of therapy systems may include three transvenous leads located as illustrated in FIGS. 1 and 3, and an additional lead located within or proximate to left atrium 38. As another example, other examples of therapy systems may include a single lead that extends from ICD 16 into right atrium 30 or right ventricle 32, or two leads that extend into a respective one of the right ventricle 32 and right atrium 30. An example of this type of therapy system is shown in FIG. 4.

FIG. 4 is a conceptual diagram illustrating another example of therapy system 80, which is similar to therapy system 10 of FIGS. 1-2, but includes two leads 18, 22, rather than three leads. Leads 18, 22 are implanted within right ventricle 32 and right atrium 30, respectively. Therapy system 80 shown in FIG. 4 may be useful for providing defibrillation and pacing pulses to heart 14. Therapy system 80 may further include INS 26 (not shown in FIG. 4), which is configured to deliver electrical stimulation therapy to one or more nerves or spinal cord 44 (FIG. 2) of patient 12 in order to help prevent or mitigate an arrhythmia of patient 12.

FIG. 5 is a functional block diagram of an example configuration of ICD 16, which includes processor 90, memory 92, signal generator 94, sensing module 96, telemetry module 98, and power source 100. Memory 92 includes computer-readable instructions that, when executed by processor 90, cause ICD 16 and processor 90 to perform various functions attributed to ICD 16 and processor 90 herein. Memory 92 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

Processor 90 may include any one or more of a microprocessor, a controller, a digital signal processor (DSP), an application specific integrated circuit (ASIC), a field-programmable gate array (FPGA), or equivalent discrete or integrated logic circuitry. In some examples, processor 90 may include multiple components, such as any combination of one or more microprocessors, one or more controllers, one or more DSPs, one or more ASICs, or one or more FPGAs, as well as other discrete or integrated logic circuitry. The functions attributed to processor 90 herein may be embodied as software, firmware, hardware or any combination thereof. Processor 90 controls signal generator 94 to deliver stimulation therapy to heart 14 according to a selected one or more of therapy programs, which may be stored in memory 92. Specifically, processor 44 may control signal generator 94 to deliver electrical pulses with the amplitudes, pulse widths, frequency, or electrode polarities specified by the selected one or more therapy programs.

Signal generator 94 is electrically coupled to electrodes 50, 52, 54, 56, 58, 60, 68, 72, 74, and 76, e.g., via conductors of the respective lead 18, 20, 22, or, in the case of housing electrode 68, via an electrical conductor disposed within housing 70 of ICD 16. Signal generator 94 is configured to generate and deliver electrical stimulation therapy to heart 14. For example, signal generator 94 may deliver defibrillation shocks to heart 14 via at least two of electrodes 68, 72, 74, 76, e.g., in a unipolar or bipolar configuration. Signal generator 94 may deliver pacing pulses via housing electrode 68, ring electrodes 50, 54, 58 coupled to leads 18, 20, and 22, respectively, and/or helical electrodes 52, 56, and 60 of leads 18, 20, and 22, respectively. In some examples, signal generator 94 delivers pacing, cardioversion, or defibrillation stimulation in the form of electrical pulses. In other examples, signal generator may deliver one or more of these types of stimulation in the form of other signals, such as sine waves, square waves, or other substantially continuous time signals.

Signal generator 94 may include a switch module and processor 90 may use the switch module to select, e.g., via a data/address bus, which of the available electrodes are used to deliver defibrillation pulses or pacing pulses. The switch module may include a switch array, switch matrix, multiplexer, or any other type of switching device suitable to selectively couple stimulation energy to selected electrodes. In other examples, however, signal generator 94 may independently deliver stimulation to electrodes 50, 52, 54, 56, 58, 60, 68, 72, 74, and 76, or selectively sense via one or more of electrodes 50, 52, 54, 56, 58, 60, 68, 72, 74, and 76, without a switch matrix.

As described in further detail below, signal generator 94 may also generate an electrical signal between two or more electrodes 50, 52, 54, 56, 58, 60, 68, 72, 74, and 76 in order to measure an electrical parameter value indicative of an impedance, e.g., of an electrical path between ICD 16 and INS 26, or to generate nontherapeutic signals for communicating with INS 26.

Sensing module 96 monitors signals from at least one of electrodes 50, 52, 54, 56, 58, 60, 68, 72, 74, and 76 in order to monitor electrical activity of heart 14, e.g., via electrogram (EGM) and/or electrocardiogram (ECG) signals. Sensing module 96 may also include a switch module to select which of the available electrodes are used to sense the heart activity. In some examples, processor 90 may select the electrodes that function as sense electrodes via the switch module within sensing module 96, e.g., by providing signals via a data/address bus. In some examples, sensing module 96 includes one or more sensing channels, each of which may comprise an amplifier. In response to the signals from processor 90, the switch module of within sensing module 96 may couple the outputs from the selected electrodes to one of the sensing channels.

In some examples, one channel of sensing module 96 may include an R-wave amplifier that receives signals from electrodes 50 and 52, which are used for pacing and sensing in right ventricle 32 of heart 14. Another channel may include another R-wave amplifier that receives signals from electrodes 54 and 56, which are used for pacing and sensing proximate to left ventricle 36 of heart 14. In some examples, the R-wave amplifiers may take the form of an automatic gain controlled amplifier that provides an adjustable sensing threshold as a function of the measured R-wave amplitude of the heart rhythm.

In addition, in some examples, one channel of sensing module 96 may include a P-wave amplifier that receives signals from electrodes 58 and 60, which are used for pacing and sensing in right atrium 30 of heart 14. In some examples, the P-wave amplifier may take the form of an automatic gain controlled amplifier that provides an adjustable sensing threshold as a function of the measured P-wave amplitude of the heart rhythm. Examples of R-wave and P-wave amplifiers are described in U.S. Pat. No. 5,117,824 to Keimel et al., which issued on Jun. 2, 1992 and is entitled, “APPARATUS FOR MONITORING ELECTRICAL PHYSIOLOGIC SIGNALS,” and is incorporated herein by reference in its entirety. Other amplifiers may also be used. Furthermore, in some examples, one or more of the sensing channels of sensing module 96 may be selectively coupled to housing electrode 68, or elongated electrodes 72, 74, or 76, with or instead of one or more of electrodes 50, 52, 54, 56, 58 or 60, e.g., for unipolar sensing of R-waves or P-waves in any of chambers 30, 32, or 36 of heart 14.

In some examples, sensing module 96 includes a channel that comprises an amplifier with a relatively wider pass band than the R-wave or P-wave amplifiers. Signals from the selected sensing electrodes that are selected for coupling to this wide-band amplifier may be provided to a multiplexer, and thereafter converted to multi-bit digital signals by an analog-to-digital converter for storage in memory 92 as an EGM. In some examples, the storage of such EGMs in memory 92 may be under the control of a direct memory access circuit. Processor 90 may employ digital signal analysis techniques to characterize the digitized signals stored in memory 92 to detect and classify the patient's heart rhythm from the electrical signals. Processor 90 may detect and classify the heart rhythm of patient 12 by employing any of the numerous signal processing methodologies known in the art. For example, processor 90 may employ signal processing methodologies to determine the heart rate and heart rate variability from the electrical signals.

Sensing module 96 is also configured to collect, measure and/or calculate impedance data for any of a variety of electrical paths that include two or more of electrodes 50, 52, 54, 56, 58, 60, 68, 72, 74, and 76. For example, sensing module 96 may collect, measure and/or calculate impedance data between housing electrode 68 and one or more of electrodes 50, 52, 54, 58, 60, 72, 74 and 76. In addition, sensing module 96 may be configured to collect, measure, and/or calculate impedance data for an impedance path between two or more electrodes 50, 52, 54, 56, 58, 60, 68, 72, 74, and 76 of ICD 16 and one or more electrodes of lead 28, which is coupled to INS 26, or the INS housing. For example, sensing module 96 may sense an electrical signal that is generated between two electrodes of lead 28, which is coupled to INS 26, and determine an impedance value or other electrical parameter value that indicates the impedance of the electrical path through tissue between ICD 16 and INS 26. Sensing module 96 may sense the intradevice signal generated by INS 26 via a wide band channel, transmit the sensed signal through an analog-to-digital converter, and then digitally process the signal with processor 90 to determine a transthoracic impedance or to extract information communicated by INS 26 via the sensed signal. Example systems and techniques for measuring interdevice impedance are described in U.S. Provisional Patent Application Ser. No. 61/110,117 to John Burnes et al., which is entitled, “INTERDEVICE IMPEDANCE,” was filed on Oct. 31, 2008, and U.S. patent application Ser. No. 12/362,895 by John Burnes et al., which is entitled, “INTERDEVICE IMPEDANCE,” was filed on Jan. 30, 2009, both of which are incorporated herein by reference in their entireties. An example IMD for determining pulmonary edema (e.g., a lung wetness status) is found in an IMD such as the Medtronic CONCERTO™, sold by Medtronic, Inc. of Minneapolis, Minn.

Sensing module 96 may also receive signals from any non-electrode sensors in wired and/or wireless communication with ICD 16, such as sensor 31 (FIG. 1). For example, sensing module 96 may receive signals indicative of blood pressure, nerve activity, lung function, lung condition, lung composition, bladder functional activities, urine flow, and/or bladder size. As described in further detail below, physiological parameters of patient, such as pressure, nerve activity, lung function, lung condition, lung composition, bladder functional activities, urine flow, and/or bladder size, may indicate the lung wetness status of patient 12. Accordingly, in some examples, ICD 16 and/or INS 26 may control therapy delivered based on signals received by sensing module 96.

If ICD 16 is configured to generate and deliver pacing pulses to heart 14, processor 90 may include pacer timing and control module, which may be embodied as hardware, firmware, software, or any combination thereof. The pacer timing and control module may comprise a dedicated hardware circuit, such as an ASIC, separate from other processor 90 components, such as a microprocessor, or a software module executed by a component of processor 90, which may be a microprocessor or ASIC. The pacer timing and control module may include programmable counters which control the basic time intervals associated with DDD, VVI, DVI, VDD, AAI, DDI, DDDR, VVIR, DVIR, VDDR, AAIR, DDIR and other modes of single and dual chamber pacing.

Intervals defined by the pacer timing and control module within processor 90 may include atrial and ventricular pacing escape intervals, refractory periods during which sensed P-waves and R-waves are ineffective to restart timing of the escape intervals, and the pulse widths of the pacing pulses. As another example, the pace timing and control module may define a blanking period, and provide signals from sensing module 96 to blank one or more channels, e.g., amplifiers, for a period during and after delivery of electrical stimulation to heart 14. The durations of these intervals may be determined by processor 90 in response to stored data in memory 92. The pacer timing and control module of processor 90 may also determine the amplitude of the cardiac pacing pulses.

During pacing, escape interval counters within the pacer timing/control module of processor 90 may be reset upon sensing of R-waves and P-waves. Signal generator 94 may include pacer output circuits that are coupled, e.g., selectively by a switching module, to any combination of electrodes 50, 52, 54, 56, 58, 60, 68, 72, 74, and 76 appropriate for delivery of a bipolar or unipolar pacing pulse to one of the chambers of heart 14. Processor 90 may reset the escape interval counters upon the generation of pacing pulses by signal generator 94, and thereby control the basic timing of cardiac pacing functions, including anti-tachyarrhythmia pacing.

The value of the count present in the escape interval counters when reset by sensed R-waves and P-waves may be used by processor 90 to measure the durations of R-R intervals, P-P intervals, P-R intervals and R-P intervals, which are measurements that may be stored in memory 92. Processor 90 may use the count in the interval counters to detect a tachyarrhythmia event, such as ventricular fibrillation event or ventricular tachycardia event. Upon detecting a threshold number of tachyarrhythmia events, processor 90 may identify the presence of a tachyarrhythmia episode, such as a ventricular fibrillation episode, a ventricular tachycardia episode, or a non-sustained tachycardia (NST) episode. Examples of tachyarrhythmia episodes that may qualify for delivery of responsive therapy include a ventricular fibrillation episode or a ventricular tachyarrhythmia episode. In the case of a NST, however, processor 90 may not meet the requirements for triggering a therapeutic response.

In some examples, processor 90 may operate as an interrupt driven device, and is responsive to interrupts from pacer timing and control module, where the interrupts may correspond to the occurrences of sensed P-waves and R-waves and the generation of cardiac pacing pulses. Any necessary mathematical calculations to be performed by processor 90 and any updating of the values or intervals controlled by the pacer timing and control module of processor 90 may take place following such interrupts. A portion of memory 92 may be configured as a plurality of recirculating buffers, capable of holding series of measured intervals, which may be analyzed by processor 90 in response to the occurrence of a pace or sense interrupt to determine whether heart 14 of patient 12 is presently exhibiting atrial or ventricular tachyarrhythmia.

In some examples, an arrhythmia detection method may include any suitable tachyarrhythmia detection algorithms. In one example, processor 90 may utilize all or a subset of the rule-based detection methods described in U.S. Pat. No. 5,545,186 to Olson et al., entitled, “PRIORITIZED RULE BASED METHOD AND APPARATUS FOR DIAGNOSIS AND TREATMENT OF ARRHYTHMIAS,” which issued on Aug. 13, 1996, or in U.S. Pat. No. 5,755,736 to Gillberg et al., entitled, “PRIORITIZED RULE BASED METHOD AND APPARATUS FOR DIAGNOSIS AND TREATMENT OF ARRHYTHMIAS,” which issued on May 26, 1998. U.S. Pat. No. 5,545,186 to Olson et al. and U.S. Pat. No. 5,755,736 to Gillberg et al. are incorporated herein by reference in their entireties. However, other arrhythmia detection methodologies may also be employed by processor 90 in other examples.

In the examples described herein, processor 90 may identify the presence of an atrial or ventricular tachyarrhythmia episode by detecting a series of tachyarrhythmia events (e.g., R-R or P-P intervals having a duration less than or equal to a threshold) of an average rate indicative of tachyarrhythmia or an unbroken series of short R-R or P-P intervals. The thresholds for determining the R-R or P-P interval that indicates a tachyarrhythmia event may be stored within memory 92 of ICD 16. In addition, the number of tachyarrhythmia events that are detected to confirm the presence of a tachyarrhythmia episode may be stored as a number of intervals to detect (NID) threshold value in memory 92. In some examples, processor 90 may also identify the presence of the tachyarrhythmia episode by detecting a variable coupling interval between the R-waves of the heart signal. For example, if the interval between successive tachyarrhythmia events varies by a particular percentage or the differences between the coupling intervals are higher than a given threshold over a predetermined number of successive cycles, processor 90 may determine that the tachyarrhythmia is present.

If processor 90 detects an atrial or ventricular tachyarrhythmia based on signals from sensing module 96, and an anti-tachyarrhythmia pacing regimen is desired, timing intervals for controlling the generation of anti-tachyarrhythmia pacing therapies by signal generator 94 may be loaded by processor 90 into the pacer timing and control module to control the operation of the escape interval counters therein and to define refractory periods during which detection of R-waves and P-waves is ineffective to restart the escape interval counters.

If ICD 16 is configured to generate and deliver defibrillation pulses to heart 14, signal generator 94 may include a high voltage charge circuit and a high voltage output circuit. In the event that generation of a cardioversion or defibrillation pulse is required, processor 90 may employ the escape interval counter to control timing of such cardioversion and defibrillation pulses, as well as associated refractory periods. In response to the detection of atrial or ventricular fibrillation or tachyarrhythmia requiring a cardioversion pulse, processor 90 may activate a cardioversion/defibrillation control module, which may, like pacer timing and control module, be a hardware component of processor 90 and/or a firmware or software module executed by one or more hardware components of processor 90. The cardioversion/defibrillation control module may initiate charging of the high voltage capacitors of the high voltage charge circuit of signal generator 94 under control of a high voltage charging control line.

Processor 90 may monitor the voltage on the high voltage capacitor, e.g., via a voltage charging and potential (VCAP) line. In response to the voltage on the high voltage capacitor reaching a predetermined value set by processor 90, processor 90 may generate a logic signal that terminates charging. Thereafter, timing of the delivery of the defibrillation or cardioversion pulse by signal generator 94 is controlled by the cardioversion/defibrillation control module of processor 90. Following delivery of the fibrillation or tachycardia therapy, processor 90 may return signal generator 94 to a cardiac pacing function and await the next successive interrupt due to pacing or the occurrence of a sensed atrial or ventricular depolarization.

Signal generator 94 may deliver cardioversion or defibrillation pulses with the aid of an output circuit that determines whether a monophasic or biphasic pulse is delivered, whether housing electrode 68 serves as cathode or anode, and which electrodes are involved in delivery of the cardioversion or defibrillation pulses. Such functionality may be provided by one or more switches or a switching module of signal generator 94.

Telemetry module 98 includes any suitable hardware, firmware, software or any combination thereof for communicating with another device, such as programmer 24 (FIG. 1). Under the control of processor 90, telemetry module 98 may receive downlink telemetry from and send uplink telemetry to programmer 24 with the aid of an antenna, which may be internal and/or external. Processor 90 may provide the data to be uplinked to programmer 24 and the control signals for the telemetry circuit within telemetry module 98, e.g., via an address/data bus. In some examples, telemetry module 98 may provide received data to processor 90 via a multiplexer.

In some examples, processor 90 may transmit atrial and ventricular heart signals (e.g., EGM and/or ECG signals) produced by atrial and ventricular sense amp circuits within sensing module 96 to programmer 24. The atrial and ventricular heart signals, as well as other physiological parameters of patient 12, e.g., transthoracic impedance, sensed by ICD 16 may be transmitted to programmer 24 or another device for diagnostic purposes, e.g., to diagnose a severity of the patient's condition. Programmer 24 may interrogate ICD 16 to receive the heart signals. Processor 90 may store heart signals within memory 92, and retrieve stored heart signals from memory 92. Processor 90 may also generate and store marker codes indicative of different cardiac episodes that sensing module 96 detects, and transmit the marker codes to programmer 24. An example pacemaker with marker-channel capability is described in U.S. Pat. No. 4,374,382 to Markowitz, entitled, “MARKER CHANNEL TELEMETRY SYSTEM FOR A MEDICAL DEVICE,” which issued on Feb. 15, 1983 and is incorporated herein by reference in its entirety.

The various components of ICD 16 are coupled to power source 100, which may include a rechargeable or non-rechargeable battery. A non-rechargeable battery may be selected to last for several years, while a rechargeable battery may be inductively charged from an external device, e.g., on a daily or weekly basis.

In some examples, data from sensing module 96 may be uploaded to a remote server, from which a clinician or another user may access the data to determine whether a potential sensing integrity issue exists or whether the measured electrical parameter value indicative of transthoracic impedance of patient 12 indicates patient 12 requires medical attention. An example of a remote server includes the CareLink Network, available from Medtronic, Inc. of Minneapolis, Minn. An example communication system that includes an external device, such as a server, and one or more computing devices that are coupled to ICD 16 and programmer 24 via a network is described below with respect to FIG. 9.

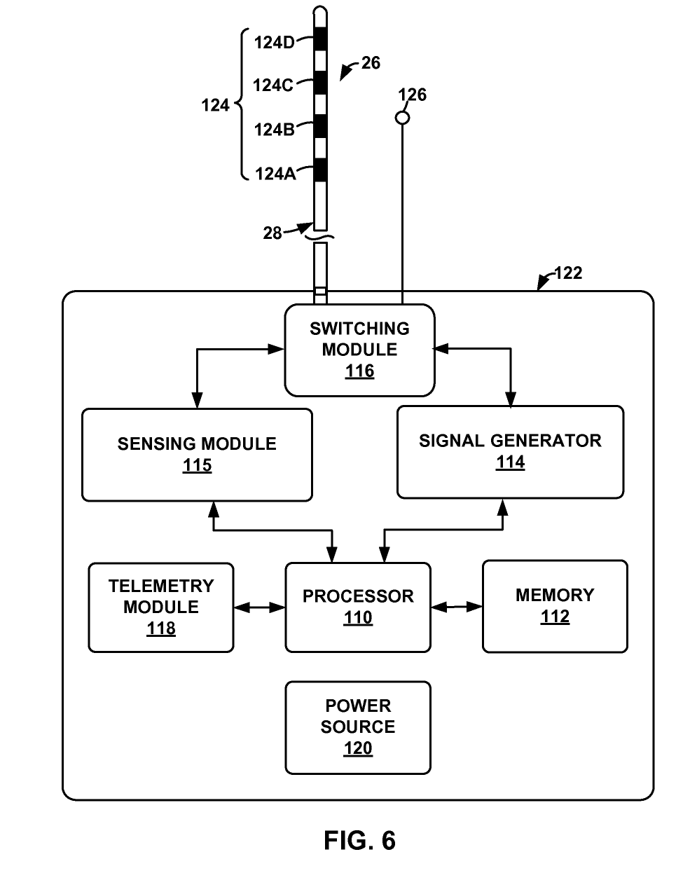

FIG. 6 is a functional block diagram of an example INS 26. INS 26 includes processor 110, memory 112, signal generator 114, sensing module 115, switching module 116, telemetry module 118, and power source 120. In the example shown in FIG. 6, processor 110, memory 112, signal generator 114, switching module 116, telemetry module 118, and power source 120 are enclosed within housing 122, which may be, for example a hermetic housing. As shown in FIG. 6, signal generator 114 is coupled to lead 28 either directly or indirectly (e.g., via a lead extension). Alternatively, signal generator 114 may be coupled more than one lead directly or indirectly (e.g., via a lead extension, such as a bifurcating lead extension that may electrically and mechanically couple to two leads) as needed to provide neurostimulation therapy to patient 12.

In the example illustrated in FIG. 6, lead 28 includes electrodes 124A-124D (collectively referred to as “electrodes 124”). Electrodes 124 may comprise ring electrodes. In other examples, electrodes 124 may be arranged in a complex electrode array that includes multiple non-contiguous electrodes at different angular positions about the outer circumference of lead 28, as well as different levels of electrodes spaced along a longitudinal axis of lead 28. The configuration, type, and number of electrodes 124 illustrated in FIG. 6 are merely exemplary. In other examples, INS 26 may be coupled to any suitable number of leads with any suitable number and configuration of electrodes. Moreover, lead 28 may comprise a shape other than a cylindrical shape. As an example, lead 28 may comprise a paddle-shaped portion that carries electrodes 124.

In some examples, as illustrated in FIG. 6, INS 26 includes one or more housing electrodes, such as housing electrode 126, which may be formed integrally with an outer surface of hermetically-sealed housing 122 of INS 26 or otherwise coupled to housing 122. In some examples, housing electrode 126 is defined by an uninsulated portion of an outward facing portion of housing 122 of INS 26. Other division between insulated and uninsulated portions of housing 122 may be employed to define two or more housing electrodes. In some examples, housing electrode 126 comprises substantially all of housing 122.

Memory 112 includes computer-readable instructions that, when executed by processor 110, cause INS 26 to perform various functions. Memory 112 may include any volatile, non-volatile, magnetic, optical, or electrical media, such as a RAM, ROM, NVRAM, EEPROM, flash memory, or any other digital media. Memory 112 may store therapy programs, which may be stored in therapy program groups, and operating instructions. The therapy programs may define a particular program of therapy in terms of respective values for electrical stimulation parameters, such as electrode combination, electrode polarity, current or voltage amplitude, pulse width and pulse rate. A program group may comprise a plurality of therapy programs that may be delivered together on an overlapping or non-overlapping basis. The stored operating instructions may guide the general operation of INS 26 under control of processor 110.

Signal generator 114 may generate stimulation signals, which may be pulses as primarily described herein, or continuous time signals, such as square or sine waves, for delivery to patient 12 via selected combinations of electrodes 124, 126. In addition, signal generator 114 may also generate an electrical signal between two or more electrodes 124, 126 in order to measure an electrical parameter value indicative of impedance, e.g., of an electrical path between ICD 16 and INS 26, or to generate signals for communicating with INS 26.

Processor 110 controls signal generator 114 according to stored therapy programs and/or program groups in memory 112 to apply particular stimulation parameter values specified by one or more of programs, such as amplitude, pulse width, and pulse rate. Processor 110 may include any one or more microprocessors, controllers, a DSPs, ASICs, FPGAs, or equivalent discrete or integrated digital or analog logic circuitry, and the functions attributed to processor 110 herein may be embodied as software, firmware, hardware or any combination thereof.

Processor 110 may control signal generator 114 to deliver stimulation according to one or more programs and/or program groups stored in memory 112 in response to a sensed parameter indicative of lung wetness. In some examples, sensing module 115 of INS 26 and/or sensing module 96 of ICD 16 may sense one or more parameters indicative of lung wetness and provide an indication of the sensed parameter to processor 110. In other examples, processor 110 of INS 26 may receive an indication of a lung wetness status (also referred to as a “lung wetness status”) of patient 12 from a sensing device that is separate from ICD 16 and INS 26, such as sensor 31 (FIG. 1). Processor 110 may receive the indication via wired or wireless communication with the sensing device.

Processor 110 may determine (e.g., identify or detect) a change in lung wetness and control signal generator 114 to deliver stimulation to patient 12 based on the lung wetness status of patient 12. As described in further detail below, in some examples, the lung wetness state of patient 12 may be determined based on a comparison of the thoracic impedance of patient 12 with a threshold value, which may be stored in memory 112. The threshold value that indicates a lung wetness status for which neurostimulation therapy is desirable may be specific to patient 12 or may be based on more than one patient. In some examples, the threshold value may be determined during implant of the therapy system within patient 12 or when patient 12 is known to be in a lung wetness state in which mitigation of the lung wetness is desirable.

Processor 110 may also receive indications of the patient's response to the stimulation from sensing module 115 of INS 26, sensing module 96 of ICD 16, and/or sensor 31, and control signal generator 114 to deliver a modified stimulation signal in response to the indicated patient response. For example, pressure, heart rate, heart rate variability, nerve activity, lung function, lung condition, lung composition, bladder functional activities, urine flow, tissue impedance, and/or bladder size may sense one or more physiological parameters indicative of lung wetness and/or potential side effects of neurostimulation, and processor 110 may control signal generator 114 to deliver a modified stimulation signal in response to continued detection of lung wetness and/or detection of neurostimulation side effects.

Signal generator 114 and sensing module 115 are coupled to switching module 116. Processor 110 may control switching module 116 to apply the stimulation signals generated by signal generator 114 to selected combinations of electrodes 124, 126. In particular, switching module 116 couples stimulation signals to selected conductors within leads 28 which, in turn, deliver the stimulation signals across selected electrodes 124, e.g., in a unipolar configuration with housing electrode 126 or a multipolar configuration. In addition, in some examples, processor 110 may control switching module 116 to connect a selected combination of electrodes 124, 126 to sensing module 115 to sense electrical signals. The electrical signals may be, for example, a far field signal generated between electrodes 50, 52, 54, 56, 58, 60, 68, 72, 74, and 76 of leads 18, 20, 22 that are coupled to ICD 16. Switching module 116 may be a switch array, switch matrix, multiplexer, or any other type of switching device suitable to selectively couple stimulation energy to selected electrodes. Hence, signal generator 114 is coupled to electrodes 124, 126 via switching module 116 and conductors within leads 28. In some examples, INS 26 does not include switching module 116.

Signal generator 114 may be a single- or multi-channel signal generator. In particular, signal generator 114 may be capable of delivering, a single stimulation pulse, multiple stimulation pulses, or a continuous signal at a given time via a single electrode combination or multiple stimulation pulses at a given time via multiple electrode combinations. In some examples, however, signal generator 114 and switching module 116 may be configured to deliver multiple channels on a time-interleaved basis. In this case, switching module 116 serves to time division multiplex the output of signal generator 114 across different electrode combinations at different times to deliver multiple programs or channels of stimulation energy to patient 12.

Sensing module 115 is configured to collect, measure and/or calculate impedance data for any of a variety of electrical paths that include two or more of electrodes 124, 126. For example, sensing module 115 may collect, measure and/or calculate impedance data between housing electrode 126 and one or more of electrodes 124 of lead 26. Processor 90 may additionally or alternatively collect, measure and/or calculate impedance data for any of a variety of electrical paths that include two or more of electrodes 124. In addition, in some examples, sensing module 115 is configured to collect, measure, and/or calculate impedance data for an impedance path between two or more electrodes 124 and two or more electrodes 50, 52, 54, 56, 58, 60, 68, 72, 74, and 76 of leads 18, 20, 22 that are coupled to ICD 16. In some examples, sensing module 115 may also be configured to monitor signals from at least one of electrodes 124 in order to monitor physiological parameters of patient 12, such as EGM/ECG signals of heart 14 (FIG. 1). Sensing module 115 may also monitor signals from physically separate sensors that are in wired and/or wireless communication with INS 24, such as sensor 31. For example, sensing module 115 may receive signals indicative of thoracic impedance, blood pressure, nerve activity, lung function, lung condition, lung composition, bladder functional activities, urine flow, and/or bladder size from sensor 31. INS 26 and/or ICD 16 may control therapy delivered based on signals received by sensing module 115.

Telemetry module 118 supports wireless communication between INS 26 and an external programmer 24 (FIG. 1) or another computing device under the control of processor 110. Processor 110 of INS 26 may receive, as updates to programs, values for various stimulation parameters such as amplitude and electrode combination, from programmer 24 via telemetry module 118. The updates to the therapy programs may be stored within memory 112.

The various components of INS 26 are coupled to power supply 120, which may include a rechargeable or non-rechargeable battery or a supercapacitor. A non-rechargeable battery may be selected to last for several years, while a rechargeable battery may be inductively charged from an external device, e.g., on a daily or weekly basis. In other examples, power supply 120 may be powered by proximal inductive interaction with an external power supply carried by patient 12.

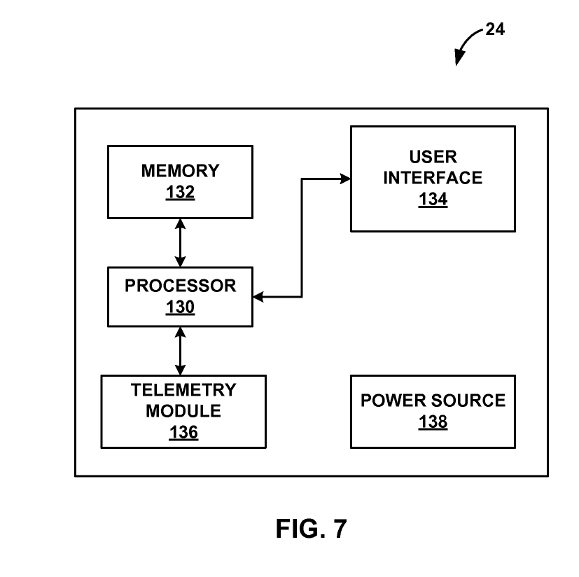

FIG. 7 is a block diagram of an example programmer 24. As shown in FIG. 7, programmer 24 includes processor 130, memory 132, user interface 134, telemetry module 136, and power source 138. Programmer 24 may be a dedicated hardware device with dedicated software for programming of ICD 16 and INS 26. Alternatively, programmer 24 may be an off-the-shelf computing device running an application that enables programmer 24 to program ICD 16 and INS 26. In some examples, separate programmers may be used to program ICD 16 and INS 26. However, a common programmer 24 that is configured to program both ICD 16 and INS 26 may provide a more streamlined programming process for a user, such as a clinician or patient 12.

A user may use programmer 24 to select therapy programs (e.g., sets of stimulation parameters), generate new therapy programs, modify therapy programs through individual or global adjustments or transmit the new programs to a medical device, such as ICD 16 or INS 26 (FIG. 1). The clinician may interact with programmer 24 via user interface 134, which may include display to present graphical user interface to a user, and a keypad or another mechanism for receiving input from a user.

Processor 130 can take the form one or more microprocessors, DSPs, ASICs, FPGAs, programmable logic circuitry, or the like, and the functions attributed to processor 130 herein may be embodied as hardware, firmware, software or any combination thereof. Memory 132 may store instructions that cause processor 130 to provide the functionality ascribed to programmer 24 herein, and information used by processor 130 to provide the functionality ascribed to programmer 24 herein. Memory 132 may include any fixed or removable magnetic, optical, or electrical media, such as RAM, ROM, CD-ROM, hard or floppy magnetic disks, EEPROM, or the like. Memory 132 may also include a removable memory portion that may be used to provide memory updates or increases in memory capacities. A removable memory may also allow patient data to be easily transferred to another computing device, or to be removed before programmer 24 is used to program therapy for another patient. Memory 132 may also store information that controls therapy delivery by ICD 16 and INS 26, such as stimulation parameter values.

Programmer 24 may communicate wirelessly with ICD 16 and INS 24, such as using RF communication or proximal inductive interaction. This wireless communication is possible through the use of telemetry module 136, which may be coupled to an internal antenna or an external antenna. An external antenna that is coupled to programmer 24 may correspond to the programming head that may be placed over ICD 16 or INS 24, as described above with reference to FIG. 1. Telemetry module 136 may be similar to telemetry module 98 of ICD 16 (FIG. 5) or telemetry module 118 of INS 26 (FIG. 6).

Telemetry module 136 may also be configured to communicate with another computing device via wireless communication techniques, or direct communication through a wired connection. Examples of local wireless communication techniques that may be employed to facilitate communication between programmer 24 and another computing device include RF communication according to the 802.11 or Bluetooth specification sets, infrared communication, e.g., according to the IrDA standard, or other standard or proprietary telemetry protocols. In this manner, other external devices may be capable of communicating with programmer 24 without needing to establish a secure wireless connection.

Power source 138 delivers operating power to the components of programmer 24. Power source 138 may include a battery and a power generation circuit to produce the operating power. In some examples, the battery may be rechargeable to allow extended operation. Recharging may be accomplished by electrically coupling power source 138 to a cradle or plug that is connected to an alternating current (AC) outlet. In addition or alternatively, recharging may be accomplished through proximal inductive interaction between an external charger and an inductive charging coil within programmer 24. In other examples, traditional batteries (e.g., nickel cadmium or lithium ion batteries) may be used. In addition, programmer 24 may be directly coupled to an alternating current outlet to power programmer 24. Power source 138 may include circuitry to monitor power remaining within a battery. In this manner, user interface 134 may provide a current battery level indicator or low battery level indicator when the battery needs to be replaced or recharged. In some cases, power source 138 may be capable of estimating the remaining time of operation using the current battery.

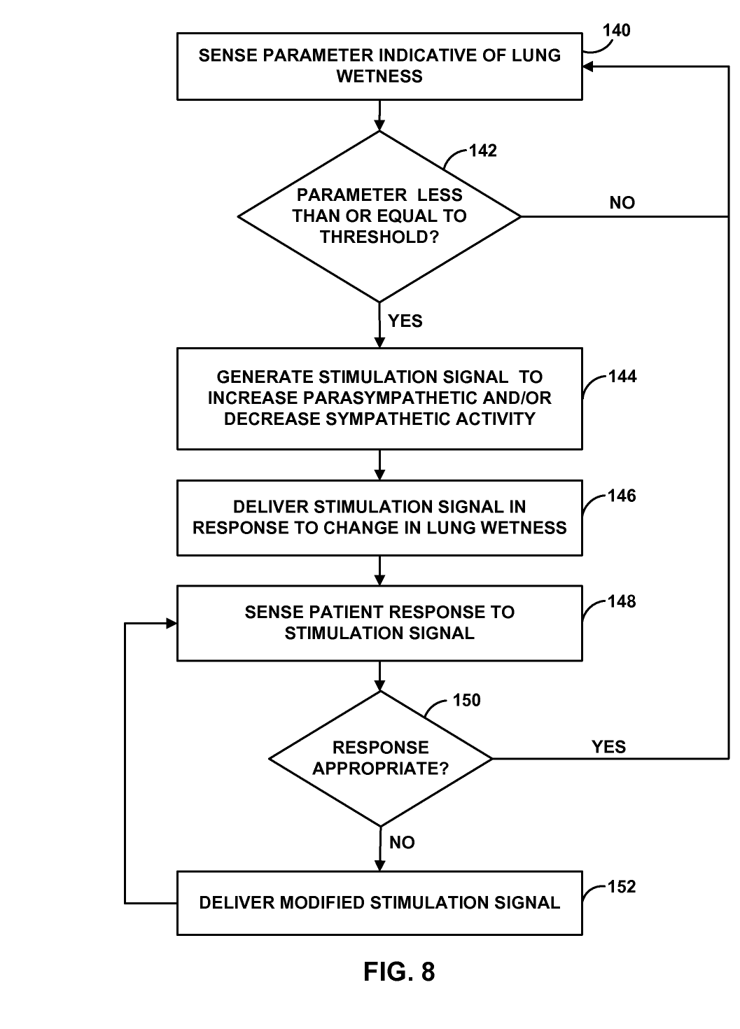

FIG. 8 is a flow diagram of an example technique for closed-loop delivery of neurostimulation to patient 12 to mitigate lung wetness. ICD 16 and/or INS 26 may sense one or more physiological parameters indicative of lung wetness within patient 12 (140). In the example shown in FIG. 8, the physiological parameter indicative of lung wetness may include thoracic impedance, which may vary as a function of fluid accumulation in the lungs. In other examples, ICD 16 and/or INS 26 may sense other parameters in addition to or as an alternative to thoracic impedance. As one example, ICD 16 and/or INS 26 may sense the posture of patient 12 in addition to thoracic impedance. The sensed thoracic impedance may vary with the posture of patient 12, and, accordingly, sensing both impedance and posture may help assure that changes in the sensed impedance are attributable to changes in lung wetness. For example, thoracic impedance may briefly decrease when patient 12 lies down and briefly increase as patient 12 stands up. Monitoring both posture and thoracic impedance may allow changes in thoracic impedance attributable to posture changes to be factored out of lung wetness detection.

As other examples, ICD 16 and/or INS 26 may sense heart rate, respiration, and/or activity in combination with thoracic impedance. Thoracic impedance may vary with each of the heart rate, respiration rate, and activity level of patient 12. Accordingly, sensing one or more of heart rate, respiration, or activity of patient 12 may help assure that changes in sensed impedance are attributable to changes in lung wetness. Processor 110 may perform signal processing or other analysis of the sensed signals to help detect changes in lung wetness. As one example, processor 110 may filter out variations in the sensed impedance signal that may be attributable to heart rate, breathing, and/or other variations in the impedance signal that are not attributable to variations in lung wetness.

Based on the one or more sensed physiological parameters, processor 110 of INS 26 may detect a change in lung wetness. For example, as shown in FIG. 8, processor 110 may determine whether a sensed intrathoracic impedance that is less than or equal to a threshold value (142). The threshold value may be stored in memory 112 of INS 26 or a memory of another device (e.g., ICD 16 or programmer 24). As another example, processor 110 may detect a decrease in intrathoracic impedance that is greater than or equal to a threshold, e.g., a threshold that defines a percentage change in a parameter value. As previously indicated, the threshold value may be specific to patient 12 or may be general to more than one patient. The threshold value may be a physiological parameter value (or a range of values) that is determined, e.g., when patient 12 is known to be in a wetness state in which mitigation of the lung wetness is desirable.

Instead or in addition to comparing a sensed physiological parameter value to a threshold value, processor 110 may detect changes in lung wetness that merit the delivery of neurostimulation therapy by analyzing trends in a plurality of signals. As one example, processor 110 may identify an increase in lung wetness if the heart rate of patient 12 increases, e.g., a threshold amount or above a threshold value, and thoracic impedance decreases, e.g., a threshold amount or below a threshold value. As another example, processor 110 may identify a threshold increase in lung wetness if the patient's respiration rate increases, e.g., a threshold amount or above a threshold value, and thoracic impedance decreases, e.g., a threshold amount or below a threshold value. Processor 110 may also examine ratios between different types of physiological parameters to detect threshold changes in lung wetness.

In other examples, processor 110 monitors other physiological parameters of patient 12 that are indicative of lung wetness in addition to or instead of intrathoracic impedance. For example, processor 110 may monitor EGM and/or ECG signals, and detect a change in lung wetness status that merits the delivery of neurostimulation therapy based on EGM and/or ECG signals sensed by ICD 16. Processor 110 may process the EGM and/or ECG signals to obtain one or more cardiac parameters, which reflect fluid retention within the lungs. A cardiac function may be a function of the QRS complex, the QRST segment of the cardiac cycle or portions of the QRST segment (e.g., the S-T segment). As examples, cardiac parameters may comprise the duration of the QRS complex, the amplitude of the QRS complex, the integral of the QRS complex, or the integral of the QRST segment. When patient 12 experiences increasing fluid in the lungs, the amplitudes of the QRS complex and the T-wave may decrease over several cardiac cycles, and, in some cases, the duration of the QRS complex may increase. Thus, it may be desirable for INS 26 to deliver therapy to patient 12 to mitigate lung wetness, e.g., as indicated by a shorter QRS complex duration or a increased QRS complex or T-wave amplitude. Examples of utilizing cardiac parameters to determine changes in lung wetness are described in U.S. Pat. No. 6,931,272 to Burnes et al., which issued on Aug. 16, 2005 and is entitled, “METHOD AND APPARATUS TO MONITOR PULMONARY EDEMA” and is incorporated herein by reference in its entirety. Processor 110 may detect threshold changes in lung wetness based on cardiac parameters alone or in combination with other parameters indicative of lung wetness, such as thoracic impedance.

As another example, processor 110 may monitor lungs sounds, e.g., via an implantable microphone implanted in patient 12 proximate to the lungs or an acoustic sensor, and detect a change in lung wetness status that merits the delivery of neurostimulation therapy based on the sensed lung sounds. The signal sensed from the lung sound sensors (referred to as the “lung sound signal”) may be filtered to provide an indication of lung wetness. In some examples, processor 110 may compare the filtered signal indicative of lung wetness sounds to a stored template in order to determine whether the sensed lung sound is indicative of a lung wetness status that merits the delivery of neurostimulation therapy.

Processor 110 may compare, for example, a slope of the amplitude of the lung sound signal over time or timing between inflection points or other critical points in the pattern of the amplitude of the lung sound signal over time to trend information. A correlation between the inflection points in the amplitude waveform of the lung sound signal or other critical points and a template may indicate a lung wetness status that merits the delivery of neurostimulation therapy. Processor 110 may implement an algorithm that recognizes a trend of the lung sound signal that characterizes such a lung wetness status. As another example, processor 110 may perform temporal correlation by sampling the lung sound signal with a sliding window and comparing the sampled waveform with a stored template waveform. If the temporal correlation between the lung sound signal and the template waveform is detected, processor 110 may determine that the sensed lung sound is indicative of a lung wetness status that merits the delivery of neurostimulation therapy.

As yet another example, processor 110 may monitor lung wetness using an implantable imaging sensor, e.g., an implantable ultrasound transducer array, to obtain imaging data indicative of lung wetness. As one example, an ultrasound transducer array may be implanted within and/or proximate to the lungs of patient 12 to record imaging data indicative of lung wetness. Processor 110 may monitor the ultrasound imaging data to detect the lung wetness status that merits the delivery of neurostimulation therapy, e.g., based on a predetermined image that is known to be indicative of such a lung wetness status. As another example, processor 110 may determine lung composition based on data obtained via ultrasonic measurements. Processor 110 may, for example, determine tissue density or fluid content with the lungs based on the ultrasound data.

As yet another example, processor 110 may monitor respiratory rate and/or respiratory volume, e.g., in combination with monitoring an activity level of patient 12 via a motion sensor. An increase in respiratory rate and/or respiratory volume without a corresponding increase in the activity level of patient 12 may indicate a deterioration of heart failure and lung wetness. An activity level of patient 12 may be monitored using any suitable technique. In some examples, processor 110 compares an amplitude or pattern of the patient activity signal generated by a motion sensor to a stored threshold or template to determine whether the patient activity level has increased. Processor 110 can also detect an increase or decrease in activity level of patient 12 between two periods of time by comparing a gross level of physical activity, e.g., activity counts based on footfalls or the like, undertaken by patient 12 during the respective periods of time. Processor 110 can determine activity counts using any suitable technique.

Suitable techniques for determining a patient's activity level or posture are described in commonly-assigned U.S. Patent Application Publication No. 2005/0209644 by Heruth et al., entitled, “COLLECTING ACTIVITY INFORMATION TO EVALUATE THERAPY,” and U.S. Patent Application Publication No. 2008/0269812 by Gerber et al., entitled, “THERAPY ADJUSTMENT.” U.S. Patent Application Publication Nos. 2005/0209644 and 2008/0269812 are incorporated herein by reference in their entireties. As described in U.S. Patent Application Publication No. 2005/0209644, a processor may determine an activity level based on a signal from a sensor, such as an accelerometer, a bonded piezoelectric crystal, a mercury switch or a gyro, by sampling the signal and determining a number of activity counts during the sample period. For example, processor 110 may compare the sample of a signal generated by a motion sensor to one or more amplitude thresholds stored within memory 112 (FIG. 6). Processor 110 may identify each threshold crossing as an activity count. Where processor 110 compares the sample to multiple thresholds with varying amplitudes, processor 110 may identify crossing of higher amplitude thresholds as multiple activity counts. Using multiple thresholds to identify activity counts, processor 110 may be able to more accurately determine changes in the patient's activity level.

Processor 110 may detect threshold changes in lung wetness based on any physiological parameter of patient 12 that is indicative of lung wetness alone or in combination with other physiological parameters of patient 12 that are indicative of lung wetness.

Returning now to the example shown in FIG. 8 in which processor 110 of INS 26 determines whether to deliver therapy to patient 12 based on sensed intrathoracic impedance, upon determining that the intrathoracic impedance is less than or equal to a threshold value (142), processor 110 of INS 26 may control signal generator 114 to generate a neurostimulation signal configured to at least one of increase parasympathetic activity or decrease sympathetic activity within the patient (144). Increasing parasympathetic activity and/or decreasing sympathetic activity may aid in mitigating lung wetness. INS 26 may deliver the neurostimulation signal to patient 12 via one or more electrodes 124 of lead 28 (146). In some examples, INS 26 delivers the neurostimulation signal to patient 12 for a predetermined duration of time, which may be stored in memory 112 of INS 26 or a memory of another device. The predetermined duration of time for the neurostimulation therapy may be selected, e.g., by a clinician, to be a duration of time in which patient 12 is expected to respond to the neurostimulation therapy, e.g., by improving cardiac function, renal function or other organ function to decrease lung wetness. In some examples, the duration of time may be approximately two hours.

In some examples, INS 26 may deliver a neurostimulation signal to patient 12 even if the sensed parameter is not less than or equal to the threshold value. For example, INS 26 may deliver therapy at a lower amplitude (compared to when the sensed parameter is less than or equal to the threshold value) when the sensed intrathoracic impedance is greater than the threshold value to maintain a particular lung status or to prevent further fluid retention by the patient's lungs. INS 26 may then deliver therapy at a higher intensity when the sensed parameter is less than or equal to the threshold value. Applying therapy at the lower maintenance or preventative rate may reduce the need for more aggressive therapy throughout the course of therapy delivery by therapy system. An intensity of therapy may be a function of, for example, the signal amplitude (e.g., current or voltage amplitude), signal duration (e.g., pulse width), frequency (e.g., pulse rate), duty cycle, and other stimulation parameter values.

ICD 16 and/or INS 26 may determine the patient's response to the neurostimulation signal (148). In some examples, the neurostimulation signal may comprise a plurality of neurostimulation signals, e.g., in the form of a program or program group. Sensing the patient's response to the neurostimulation signal may comprise sensing the patient's response to the neurostimulation signals throughout the duration of neurostimulation delivery. As one example, ICD 16 and/of INS 26 may monitor one or more physiological parameters indicative of lung wetness during and/or after INS 26 delivers neurostimulation therapy to patient 12. As previously described, parameters indicative of lung wetness may include intrathoracic impedance or cardiac parameters that are based on EGM/ECG signals, e.g., QRS width and/or ST segment data. As other examples, during and/or after INS 26 delivers neurostimulation therapy to patient 12, ICD 16 and/or INS 26 may monitor one or more physiological parameters indicative of an increase in cardiac output, an improvement in cardiac function, an increase renal function or other changes in organ function that may reflect a decrease in lung wetness. Examples of these physiological parameters include contractility of heart 14, heart rate, heart rate variability, heart sounds, lung sounds, respiration activity (e.g., respiration rate, inhalation duration and rate, and/or exhalation duration and rate), tissue perfusion, blood oxygen saturation, tissue temperature, blood pressure, bladder size, bladder functional activities (e.g., frequency or volume of urination), urine flow, lung function, lung composition, and/or nerve activity. The heart and lung sounds may be monitored by, for example, an implanted microphone or an acoustic sensor.

The contractility of heart 14, heart rate, and heart rate variability may provide indications of the cardiac function of heart 14. Sensing one or more of these parameters in response to neurostimulation therapy may provide an indication of how the neurostimulation is affecting and/or affected cardiac function. As described previously, ICD 16 or sensor 31 may sense electrical signal indicative of heart rate and/or heart rate variability. ICD 16, sensor 31 (FIG. 1) or another device may also sense the contractility of heart 14, for example, using a strain gauge or other pressure sensor proximate to the myocardium of heart 14. In some cases, an increase in the contractility of heart 14 may indicate the cardiac output of heart 14 has improved. In addition, a decrease in heart rate or blood pressure may also indicate an improvement in cardiac function. An improvement in cardiac function may help decrease lung wetness. Thus, a detected increase in the contractility of heart 14 or a decrease in heart rate or heart rate variability in response to the delivery of neurostimulation may indicate the neurostimulation therapy provided by INS 26 has helped mitigate lung wetness.

Blood pressure may provide an indication of autonomic tone, e.g., sympathetic and parasympathetic activity. Consequently, monitoring blood pressure may provide an indication of how neurostimulation is affecting and/or affected autonomic tone. Sensing blood pressure proximate to the target stimulation site of INS 26 may be provide a more precise measure of neurostimulation impact than monitoring systemic blood pressure. In other examples, ICD 16 and/or INS 26 may monitor blood pressure in the lungs, great veins (e.g., intravascular pressure in the superior vena cava, inferior vena cava, and/or one or more of the pulmonary veins), and/or heart (e.g., intracardiac pressure) of patient 12. In some cases, a decrease in blood pressure following the delivery of neurostimulation therapy may indicate that the cardiac function of heart 14 has improved, which may indicate the neurostimulation therapy provided by INS 26 has helped mitigate lung wetness.

Bladder size may be indicative of fluid retention by the kidneys and renal sympathetic tone. Accordingly, bladder size may be monitored (e.g., using a strain gauge or other pressure sensor proximate to a wall of the bladder or patient 12), to provide an indication of renal sympathetic activity when INS 26 delivers neurostimulation to modulate renal autonomic activity. In some cases, an increase in bladder size following the delivery of neurostimulation therapy may indicate that the fluid processing by the kidneys of patient 12 has increased. An increase in fluid processing by one or more kidneys of patient 12 may help decrease fluid retention by patient 12, and, therefore, decrease lung wetness. Thus, an increase in bladder size following the delivery of neurostimulation therapy may indicate the neurostimulation helped mitigate lung wetness.

Bladder functional activities and/or urine flow may be monitored following the delivery of neurostimulation therapy to provide an indication of a level of fluid processing by the kidneys of patient 12. Bladder functional activities and/or urine flow may provide information similar to monitoring of bladder size. For example, an increase in bladder functional activities and/or urine flow following the delivery of neurostimulation therapy may indicate the neurostimulation helped mitigate lung wetness.

Monitoring neural activity, e.g., of a sympathetic and/or parasympathetic nerve proximate to the target stimulation site of INS 26, may provide a direct measurement of sympathetic and/or parasympathetic neural activity. Sensing one or more physiological parameters prior to neurostimulation by INS 26 may provide a baseline for comparison of post-neurostimulation measurements. In some cases, memory 112 of INS 26 or another device may store threshold values for neural activity. An increase in parasympathetic neural activity following the delivery of neurostimulation to patient 12 may indicate that the neurostimulation helped mitigate lung wetness. In addition, in some cases, a decrease in sympathetic neural activity following the delivery of neurostimulation to patient 12 may indicate that the neurostimulation helped mitigate lung wetness.

Processor 110 of INS 26 may determine if the patient's response to the neurostimulation therapy is appropriate, e.g., mitigates lung wetness without substantial side effects (150). Processor 110 may make this determination during and/or after INS 26 delivers therapy. An appropriate patient response to the neurostimulation may be detected, e.g., by detecting an increase in the contractility of heart 14 or the heart rate, an increase in blood pressure, an increase in bladder size, an increase in bladder functional activities, an increase in urine flow, an increase in lung function, a decrease in fluid content within the lungs or other similar change in lung composition, an increase in parasympathetic neural activity, and/or a decrease in sympathetic neural activity following the delivery of neurostimulation to patient 12 by INS 26.

Processor 110 may also detect an appropriate patient response to the neurostimulation by detecting whether the lung wetness is still present and is still greater than a threshold value (e.g., an impedance less than or equal to a stored threshold value). For example, processor 110 may identify a decrease in contractility of heart 14 during therapy delivery by INS 26, which may indicate the neurostimulation therapy may not have mitigated lung wetness. In response to a determination that the patient's response to the neurostimulation therapy is not appropriate, e.g., resulted in side effects and/or failed to mitigate lung wetness, processor 110 may control stimulation generator 114 to deliver neurostimulation with modified parameters (152). The modified parameters may be selected based on the patient's response to the neurostimulation signals.

In some examples, memory 112 of INS 26 or another device may store a plurality of therapy programs that each defines a different neurostimulation therapy for patient 12 to mitigate lung wetness. Thus, in some examples, processor 110 modifies the neurostimulation signal by selecting at least one different therapy program from memory 112 and delivering therapy to patient 12 according to the new therapy program(s). Processor 110 may cease delivering therapy to patient 12 according to the previously-selected therapy program prior to delivering neurostimulation therapy to patient 12 according to the new therapy program.

In other examples, rather than selecting a new therapy program, processor 110 modifies the stimulation signal (152) by modifying one or more stimulation parameter values of the current therapy program. Memory 112 of INS 26 or another device may store a plurality of rules that indicate acceptable ranges for different stimulation parameters, such as amplitude (current or voltage), frequency, pulse width, and the like. Processor 110 may modify the one or more stimulation parameter values within the predetermined ranges in order to modify the stimulation signal and deliver therapy via the modified neurostimulation signal (152). In other examples, rather than modifying the stimulation signal, processor 110 of INS 26 may continue delivering stimulation therapy to patient 12 for a longer period of time.

If processor 110 determines that the patient's response to the neurostimulation signal is appropriate (150), e.g., decreases lung wetness without substantial side effects, ICD 16 and/or INS 26 may return to sensing one or more parameters indicative of lung wetness (140).

Although processor 110 is primarily described as detecting threshold changes in lung wetness with respect to the technique shown in FIG. 8, processor 90 of ICD 16, processor 130 of programmer 24, and/or any other suitable processor may, alone or in combination with processor 110, aid in determining changes in lung wetness. As one example, processor 90 of ICD 16 may identify a threshold change in lung wetness and provide an indication to INS 26 to cause INS 26 to deliver neurostimulation therapy to increase parasympathetic nerve activity or decrease sympathetic nerve activity in order to mitigate the lung wetness.

In addition, although FIG. 8, as well as the other techniques described herein for mitigating lung wetness are described with respect to therapy system 10 including both ICD 16 and INS 26, in some examples, the techniques described herein may be implemented by a therapy system including only an INS 26.

FIG. 9 is a block diagram illustrating a system 160 that includes an external device 162, such as a server, and one or more computing devices 164A-164N that are coupled to ICD 16, INS 26, and programmer 24 shown in FIG. 1 via a network 166, according to one example. In this example, ICD 16 and INS 26 uses their respective telemetry modules 98 (FIG. 5) and 118 (FIG. 6) to communicate with programmer 24 via a first wireless connection, and to communicate with an access point 168 via a second wireless connection. In the example of FIG. 9, access point 168, programmer 24, external device 162, and computing devices 164A-164N are interconnected, and able to communicate with each other, through network 166.

In some cases, one or more of access point 168, programmer 24, external device 162, and computing devices 164A-164N may be coupled to network 166 through one or more wireless connections. ICD 16, INS 26, programmer 24, external device 162, and computing devices 164A-164N may each comprise one or more processors, such as one or more microprocessors, DSPs, ASICs, FPGAs, programmable logic circuitry, or the like, that may perform various functions and operations, such as those described herein.

Access point 168 may comprise a device that connects to network 166 via any of a variety of connections, such as telephone dial-up, digital subscriber line (DSL), cellular phone network, or cable modem connections. In other examples, access point 168 may be coupled to network 166 through different forms of connections, including wired or wireless connections. In some examples, access point 168 may communicate with programmer 24, ICD 16, and/or INS 26. Access point 168 may be co-located with patient 12 (e.g., within the same room or within the same site as patient 12) or may be remotely located from patient 12. For example, access point 168 may be a home monitor that is located in the patient's home or is portable for carrying with patient 12.

During operation, ICD 16 and/or INS 26 may collect, determine, and store various forms of diagnostic data. For example, as described previously, ICD 16 or INS 26 may collect electrical parameter values indicative of lung wetness or other physiological parameters (e.g., heart rate, bladder size, bladder functional activities, urine flow, lung function, lung composition, blood pressure, and the like). In certain cases, ICD 16 or INS 26 may directly analyze collected diagnostic data and generate any corresponding reports or alerts. In some cases, however, ICD 16 or INS 26 may send the electrical parameter values indicative of lung wetness, to programmer 24, access point 168, and/or external device 162, either wirelessly or via access point 168 and network 166, for remote processing and analysis.

For example, ICD 16 may send programmer 24 collected electrical parameter values indicative of lung wetness, which is then analyzed by programmer 24. Programmer 24 may generate reports or alerts after analyzing electrical parameter values and determine whether the values indicate that patient 12 requires medical attention, e.g., based on the electrical parameter values exceeding a threshold value, thereby indicating patient 12 is retaining a relatively large amount of fluid within the lungs. In some cases, ICD 16, INS 26, and/or programmer 24 may combine all of the diagnostic data into a single displayable lung wetness report, which may be displayed on programmer 24. The lung wetness report may contain information concerning the lung wetness determinations, the time of day at which the determinations were taken, and identify any patterns in the lung wetness determinations. A clinician or other trained professional may review and/or annotate the lung wetness report, and possibly identify any patient conditions (e.g., congestive heart failure).

In another example, ICD 16 or INS 26 may provide external device 162 with collected lung wetness data via access point 168 and network 166. External device 162 includes one or more processors 170. In some cases, external device 162 may request collected lung wetness data, and in some cases, ICD 16 or INS 26 may automatically or periodically provide such data to external device 162. Upon receipt of the lung wetness data via input/output device 172, external device 162 is capable of analyzing the data and generating reports or alerts upon determination that the lung wetness data indicates a patient condition may exist.

In one example, external device 162 may combine the diagnostic data into a lung wetness report. One or more of computing devices 164A-164N may access the report through network 166 and display the report to users of computing devices 164A-164N. In some cases, external device 162 may automatically send the report via input/output device 172 to one or more of computing devices 164A-164N as an alert, such as an audio or visual alert. In some cases, external device 162 may send the report to another device, such as programmer 24, either automatically or upon request. In some cases, external device 162 may display the report to a user via input/output device 172.

In one example, external device 162 may comprise a secure storage site for diagnostic information that has been collected from ICD 16, INS 26, and/or programmer 24. In this example, network 166 may comprise an Internet network, and trained professionals, such as clinicians, may use computing devices 164A-164N to securely access stored diagnostic data on external device 162. For example, the trained professionals may need to enter usernames and passwords to access the stored information on external device 162. In one example, external device 162 may be a CareLink server provided by Medtronic, Inc., of Minneapolis, Minn.

The techniques described in this disclosure, including those attributed to ICD 16, INS 26, programmer 24, or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, image processing devices or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed to support one or more aspects of the functionality described in this disclosure.

Various examples have been described. These and other examples are within the scope of the following claims.

1. A method comprising:
sensing a physiological parameter indicative of lung wetness within a patient;
generating a neurostimulation signal configured to at least one of increase parasympathetic activity or decrease sympathetic activity within the patient to mitigate lung wetness based on the sensed physiological parameter; and
delivering the neurostimulation signal to the patient.

2. The method of claim 1, further comprising determining a change in lung wetness, wherein generating the neurostimulation signal comprises generating the neurostimulation signal in response to detecting an increase in lung wetness.

3. The method of claim 1, further comprising comparing the physiological parameter to a threshold value, wherein delivering the neurostimulation signal to the patient comprises delivering the neurostimulation signal to the patient based on the comparison.

4. The method of claim 3, wherein delivering the neurostimulation signal to the patient based on the comparison comprises delivering the neurostimulation signal to the patient if the physiological parameter is less than or equal to the threshold value.

5. The method of claim 1, wherein the physiological parameter comprises at least one of thoracic impedance, lung sounds, a parameter indicative of coughing, tissue impedance, a blood parameter, a time of day parameter, or an image of a lung of the patient.

6. The method of claim 1, further comprising determining a response of the patient to delivery of the neurostimulation signal, and delivering a modified neurostimulation signal to the patient based on the determined response of the patient.

7. The method of claim 6, wherein determining the response comprises determining the response based on the physiological parameter indicative of lung wetness.

8. The method of claim 6, wherein determining the response comprises at least one of sensing contractility or a heart rate of a heart of the patient, determining a heart rate variability, sensing blood pressure of the patient, determining a bladder size of the patient, sensing a bladder functional activity of the patient, sensing urine flow of a bladder of the patient, sensing a lung function parameter, sensing a lung composition parameter, sensing a blood parameter, sensing a time of day parameter, sensing a tissue impedance parameter, or sensing nerve activity of the patient.

9. The method of claim 1, wherein the neurostimulation signal is configured to mitigate lung wetness by improving cardiac function of a heart of the patient.

10. The method of claim 9, wherein improving cardiac function comprises increasing cardiac output.

11. The method of claim 1, wherein the neurostimulation signal is configured to mitigate lung wetness by decreasing renal sympathetic activity of the patient.

12. The method of claim 11, wherein decreasing renal sympathetic activity comprises increasing fluid excretion.

13. The method of claim 1, wherein the neurostimulation signal comprises an adjusted neurostimulation signal, the method further comprising delivering an initial neurostimulation signal to the patient, wherein sensing the physiological parameter comprising sensing the physiological parameter subsequent to delivering the initial neurostimulation signal.

14. The method of claim 1, wherein the generating the neurostimulation signal comprises generating a first neurostimulation signal that increases parasympathetic activity within the patient and generating a second neurostimulation signal that decreases sympathetic activity within the patient, wherein delivering the neurostimulation signal comprises alternating between delivering the first neurostimulation signal and the second neurostimulation signal.

15. The method of claim 1, wherein delivering the neurostimulation signal to the patient comprises delivering the neurostimulation signal to at least one of a dorsal vagal motonucleus, a nucleus ambiguus, a nucleus tractus solitarii, a hypothalamus, or a spinal intermediolateral column of a brain of the patient.

16. The method of claim 1, wherein delivering the neurostimulation signal to the patient comprises delivering the neurostimulation signal to a nerve associated with a lung of the patient.

17. A system comprising:
a sensor that senses a physiological parameter indicative of lung wetness within a patient;
a stimulation generator; and
a processor that controls the stimulation generator to generate and deliver a neurostimulation signal to the patient based on the sensed physiological parameter, wherein the neurostimulation signal is configured to at least one of increase parasympathetic activity or decrease sympathetic activity within the patient to mitigate lung wetness.

18. The system of claim 17, wherein the processor determines a change in lung wetness and controls the stimulation generator to generate and deliver the neurostimulation signal in response to detecting an increase in lung wetness.

19. The system of claim 17, wherein the processor compares the physiological parameter to a threshold value and controls the stimulation generator to generate and deliver the neurostimulation signal based on the comparison.

20. The system of claim 19, wherein the processor controls the stimulation generator to generate and deliver the neurostimulation signal if the physiological parameter is less than or equal to the threshold value.

21. The system of claim 17, wherein the physiological parameter comprises at least one of thoracic impedance, a lung composition parameter, lung sounds, a blood parameter, a time of day parameter, tissue impedance, or an image of a lung of the patient.

22. The system of claim 17, wherein the processor determines a response of the patient to delivery of the neurostimulation signal, and controls the stimulation generator to generate and deliver a modified neurostimulation signal to the patient based on the determined response of the patient to delivery of the neurostimulation signal.

23. The system of claim 22, wherein the processor determines the response based on the physiological parameter indicative of lung wetness.

24. The system of claim 23, wherein the sensor comprises a first sensor, the system further comprising a second sensor that senses at least one of contractility or a heart rate of a heart of the patient, a heart rate variability, blood pressure, bladder size of the patient, bladder functional activity of the patient, urine flow of a bladder of the patient, lung function of the patient, a lung composition parameter, a blood parameter, a time of day parameter, a tissue impedance parameter, or nerve activity of the patient, wherein the processor determines the response based on the sensed at least one of contractility, heart rate, heart rate variability, blood pressure, bladder size, bladder functional activity, urine flow, lung function, lung composition, blood parameter, time of day parameter, tissue impedance parameter or nerve activity of the patient.

25. The system of claim 17, wherein the neurostimulation signal is configured to mitigate lung wetness by improving cardiac function.

26. The system of claim 17, wherein the neurostimulation signal is configured to mitigate lung wetness by decreasing renal sympathetic activity.

27. A system comprising:
means for sensing a physiological parameter indicative of lung wetness within a patient;
means for generating a neurostimulation signal configured to at least one of increase parasympathetic activity or decrease sympathetic activity within the patient to mitigate lung wetness based on the sensed physiological parameter; and
means for delivering the neurostimulation signal to the patient.

28. The system of claim 27, further comprising means for determining a change in lung wetness, wherein the means for generating the neurostimulation signal comprises means for generating the neurostimulation signal in response to detecting an increase in lung wetness.

29. The system of claim 27, further comprising means for determining a response of the patient to the neurostimulation signal, and means for delivering a modified neurostimulation signal to the patient based on the determined response of the patient.

30. The system of claim 29, wherein the means for determining the response of the patient to the neurostimulation signal comprises at least one of:
the means for sensing the physiological parameter indicative of lung wetness within the patient;
means for sensing contractility of a heart of the patient;
means for sensing a heart rate of the patient;
means for determining a heart rate variability of the patient;
means for sensing a blood pressure of the patient;
means for determining a bladder size of the patient;
means for sensing a bladder functional activity of the patient;
means for sensing urine flow of a bladder of the patient;
means for sensing lung function of the patient;
means for sensing lung composition of the patient;
means for sensing a blood parameter;
means for sensing a time of day parameter;
means for sensing tissue impedance; or
means for sensing nerve activity of the patient.

31. A computer-readable medium comprising instructions that cause a processor to control a stimulation generator to generate and deliver a neurostimulation signal to a patient based on a sensed physiological parameter indicative of lung wetness, wherein the neurostimulation signal is configured to at least one of increase parasympathetic activity or decrease sympathetic activity within the patient to mitigate lung wetness.

32. A method of mitigating lung wetness of a lung of a patient, wherein the method is characterized by implanting a medical device in the patient, the medical device comprising:
a sensor that senses a physiological parameter indicative of lung wetness within a patient;
a stimulation generator; and
a processor that controls the stimulation generator to generate and deliver a neurostimulation signal to the patient based on the sensed physiological parameter, wherein the neurostimulation signal is configured to at least one of increase parasympathetic activity or decrease sympathetic activity within the patient to mitigate lung wetness.
